(19) United States
(12) Patent Application Publication
(10) Pub. No.: US 2021/0403625 A1
(43) Pub. Date: Dec. 30, 2021

(54) BORONIC ACID POLYMERS AND METHODS OF USE

(71) Applicant: GLYSCEND, INC., Baltimore, MD (US)

(72) Inventors: Thomas Henry JOZEFIAK, Belmont, MA (US); Ashish NIMGAONKAR, Ellicott City, MD (US); Steven C. POLOMOSCANIK, Methuen, MA (US)

(21) Appl. No.: 17/277,702

(22) PCT Filed: Sep. 20, 2019
(86) PCT No.: PCT/US2019/052110

§ 371 Date: Mar. 18, 2021

Related U.S. Application Data
(60) Provisional application No. 62/734,054, filed on Sep. 20, 2018.

(51) Int. Cl. C08F 230/06; C08L 43/00; A61K 45/06; A61P 3/08

(52) U.S. Cl. CPC C08F 230/06; A61P 3/08; A61K 45/06; C08L 43/00

(57) ABSTRACT

The disclosure relates to cationic boronic acid containing polymers and to methods of using the same to treat metabolic and gastrointestinal disorders.

### BACKGROUND OF THE INVENTION

Type-2 diabetes mellitus (T2DM) affects about 11.3% of the U.S. adult population, with 35% of the U.S. adults having pre-diabetic symptoms. U.S. healthcare costs due to diabetes are approaching $200 billion annually. The incidence of T2DM continues to increase in parallel with the obesity epidemic, and a major portion of present treatment for T2DM consists of a regimen of oral and intravenous medications that may be suboptimal for many subjects, in part because of side effects associated with systemic absorption of their medication. Bariatric surgery to bypass or exclude the duodenum from the digestive tract has been shown to improve T2DM. The Diabetes Surgery Summit recommends bariatric surgery to treat T2DM in some obese patients (grade III obesity), and that bariatric surgery should be considered for treatment of other patients. (See, e.g., Koliaki, C. et al., BMC Endocr Disord. (2017) 17:50 DOI 10.1186/s12902-017-0202-6.)

Analysis of the typical diabetic patient's path from first line drugs to insulin and on to surgery and other highly invasive treatments reveals striking gaps, not limited to ineffective treatments and clinical inertia. Surgery and pharmacological solutions also have failed to achieve widespread adoption. The addition of specialist clinicians in the care pathway has contributed to those failures. Accordingly, an effective treatment in the hands of the primary care physician would likely reach a much larger segment of the subject population than those which require a specialist, such as an endocrinologist, a gastroenterologist, or a surgeon, and would therefore have much greater impact.

US 2006/0134062 discloses polymers in which certain arylboronic acid moieties are bonded to the polymer backbone through hydrolytically labile linkers, and the use of such polymers as inhibitors of lipase. The polymer backbone is said to be not critical for lipase inhibition, as in this case the polymer conjugate delivers a small molecule lipase inhibitor. T2DM is not addressed.

U.S. Pat. No. 7,943,713 discloses certain polyamine boronic acid derivatives and their use to increase paper wet web strength and wet strength. The preferred polymers are characterized by aryl boronic acid moieties that are directly bonded to a carbon atom in the polymer backbone, or bonded to a carbon atom in the polymer backbone through an amide linkage. No pharmacological uses are addressed.

WO 2017/024237 discloses certain cationic polymers and use of the polymers for complexing mucus to form an occlusive barrier in the duodenum.

Seno, M. et. al, Materials Science and Engineering C, 62 (2016) 474-479, discloses certain pH- and sugar-sensitive multilayer films that are composed of phenylboronic acid-modified poly(allylamine hydrochloride) and pol(vinyl alcohol). Sato, K. et al, Langmuir, 2014, 30, 9247-9250, also discloses multilayer films that are composed of phenylboronic acid-modified poly(allylamine hydrochloride) and pol(vinyl alcohol).

A need therefore exists for new medications and non-invasive methods for treating subjects with T2MD and related metabolic disorders.

### SUMMARY OF THE INVENTION

The invention provides polymer compositions for forming a physical barrier in the gastrointestinal (GI) tract of a subject between the intestinal lining and the luminal contents. The polymers of the invention are mucin-interacting agents which form a physical barrier in-situ by interaction with resident mucin in the GI tract.

The inventors have discovered that the incorporation of pendant boronic acid moieties into certain cationic polymers dramatically improved the mucin-complexing activity of the polymers. In contrast, the addition of pendant boronic acid moieties did not significantly alter the mucin-complexing activity of neutral or anionic polymers.

As shown in the examples, the polymers described herein have improved mucin and mucus complexing activity in comparison to comparable cationic polymers, and can effectively condense mucin and mucus at the pH of the duodenum. The polymers bind tightly to mucus at the pH of the duodenum and once bound to mucus are resistant to removal by high concentrations of salts (e.g., 1M NaCl) or acidic conditions (e.g., 0.02M HCl). The resulting polymer-mucus complexes have dramatically different properties in comparison to free mucus. For example, as shown herein, the polymers reduce the diffusion coefficient of small particles in mucus by about two orders of magnitude (˜100×). When administered to a rat model of diabetes, the polymers effectively reduced blood glucose levels in an oral glucose tolerance test and showed dose responsive improvements in blood glucose levels. These results replicate the improvements in glucose homeostasis and reduced insulin resistance that are observed in patients that undergo “metabolic surgery” to bypass or exclude the duodenum from the digestive tract, but unlike surgery or invasive endoscopic procedures, the polymers of this invention are easily administered in a non-invasive manner. Without wishing to be bound by any particular theory or mechanism, these data demonstrate that the polymers are capable of complexing mucus in the duodenum to form an occlusive, non-absorbed luminal barrier when orally administered. The formation of the duodenal occlusive barrier is functionally similar or equivalent to bypassing, excluding, or ablating the duodenum by surgical or endoscopic procedures, and results in improved glucose homeostasis. (See, e.g., Koliaki, C. et al., BMC Endocr Disord. (2017) 17:50 DOI 10.1186/s12902-017-0202-6.)

This disclosure relates to cationic polymers that contain pendant boronic acid groups, which are bonded directly or indirectly to the polymer backbone through an amine or amide or carbamate or thiocarbamate linking functionality, and to a method of treating metabolic diseases that include administering a therapeutically effective amount of such a polymer to a subject in need thereof.

Another aspect of the invention is a pharmaceutical composition comprising the polymers of the present invention, along with a carrier or diluent. The pharmaceutical composition can be used for therapy, such as in the treatment of a disorder described herein. Similarly, the invention provides for the use of a polymer disclosed herein as a medicament and for the use of a polymer disclosed herein in the manufacture of a medicament for the treatment of a disorder described herein.

### BRIEF DESCRIPTION OF THE DRAWINGS

FIG. 1 illustrates the mucin complexing assay.

FIG. 2 illustrates the mucin pass-through centrifuge assay.

FIG. 3 is a chart showing the results of an evaluation of co-polymers of 3-acrylamidophenyl boronic acid (APBA) with neutral (2-hydroxypropyl-methacrylamide, HPMA), anionic (2-Acrylamido-2-methyl-1-propanesulfonic acid, AMPS), and cationic ((3-methacrylamido)propylamine, MAPAn) co-monomers using the mucin pass-through centrifuge assay. Copolymers that were evaluated contained neutral, anionic and cationic monomers and 7.5%, 15% or 30% APBA. The results show that the addition of APBA to cationic polymers, but not anionic or neutral polymers, dramatically improved the mucin complexing activity of the polymer.

FIG. 4 is a chart showing the results of an evaluation of several cationic polymers and corresponding co-polymers that contained 30%, 20% APBA using the mucin pass-through centrifuge assay. The polymers tested were poly((3-methacrylamido)propylamine (MAPAn), MAPAn/APBA (70/30), poly(3-acrylamidopropyl)trimethylammonium chloride (APTAC), APTAC/APBA (70/30), poly(3-acrylamidopropyl)dimethylamine (APDMAn), APDMAn/APBA (70/30), poly4-vinylpyridine (4-VPy), 4-VPy/APBA (80/20) and 1-vinylimidazole (1-VI), 1-VI/APBA (80/20). The results show that the addition of APBA enhanced the mucin complexing activity of each cationic polymer.

FIG. 5 is a 1H-NMR spectrum of poly(allylamine hydrochloride) (PAAn-HCl) modified with 4-carboxyphenylboronic acid (CPBA). The NMR spectrum shows that the polymer has a degree of substitution of 16 mol %. No NMR signals from anticipated impurities were observed.

FIGS. 6A and 6B illustrate the results of an oral glucose tolerance test on Study Day 8 for the control and the polymer treatment groups respectively. FIG. 6A depicts the blood glucose levels over a course of 120 minutes from each group following glucose administration. FIG. 6B compares the reduction in the incremental area under the blood glucose curve for the control and treatment groups respectively.

FIGS. 7A and 7B illustrate the results of an oral glucose tolerance test on Study Day 30 for the control and the polymer treatment groups respectively. FIG. 7A depicts the blood glucose levels over a course of 210 minutes from each group following glucose administration. FIG. 7B compares the reduction in the incremental area under the blood glucose curve for the control and treatment groups respectively.

FIGS. 8A and 8B illustrate the results of an oral glucose tolerance test on Study Day 39 for the control and the polymer treatment groups respectively. FIG. 8A depicts the blood glucose levels over a course of 240 minutes from each group following glucose administration. FIG. 8B compares the reduction in the incremental area under the blood glucose curve for the control and treatment groups respectively.

FIG. 9 illustrates the results of an insulin tolerance test on Study Day 42 for the control and the polymer treatment groups respectively.

FIGS. 10A and 10B illustrate the results of a mixed meal tolerance test on Study Day 46 for the control and the polymer treatment groups respectively. FIG. 10A depicts the blood glucose levels over a course of 180 minutes from each group following glucose administration. FIG. 10B compares the reduction in the incremental area under the blood glucose curve for the control and treatment groups respectively.

FIGS. 11A and 11B illustrate the results of an oral glucose tolerance test on Study Day 52 for the control and the polymer treatment groups respectively. In all other tolerance tests illustrated, the treatment polymer was given 60-minutes prior to glucose load. For the study illustrated in FIG. 11, the last prior treatment dose was given prior to the overnight fast, 15-hours before glucose load. FIG. 11A depicts the blood glucose levels over a course of 120 minutes from each group following glucose administration. FIG. 11B compares the reduction in the incremental area under the blood glucose curve for the control and treatment groups respectively.

FIG. 12 illustrates the results of an insulin tolerance test on Study Day 55 for the control and the polymer treatment groups respectively.

FIG. 13 illustrates the normalized body weight for GK rats completing the 60-day chronic dosing study for the control and the polymer treatment groups respectively.

FIG. 14 illustrates the weight loss for GK rats completing the 60-day dosing study for the control group.

FIG. 15 compares the food intake over the study period for the control and the polymer treatment groups respectively.

FIGS. 16A-16C illustrate the results of metabolic testing conducted after week 1 of chronic GK rodent study. FIG. 16A compares the blood glucose for the control and the polymer treatment groups respectively. FIG. 16B compares the insulin levels for the control and the polymer treatment groups respectively. FIG. 16C is a homeostatic model assessment of insulin resistance for the control and the polymer treatment groups respectively.

FIGS. 17A-17C illustrate the results of metabolic testing conducted after week 8 of chronic GK rodent study. FIG. 17A compares the blood glucose for the control and the polymer treatment groups respectively. FIG. 17B compares the insulin levels for the control and the polymer treatment groups respectively. FIG. 17C is a homeostatic model assessment of insulin resistance for the control and the polymer treatment groups respectively.

### DETAILED DESCRIPTION OF THE INVENTION

This disclosure relates to cationic polymers that contain pendant boronic acid groups, which are bonded directly or indirectly to the polymer backbone through an amine or amide bond. The cationic polymers are preferably polycations that include amine- or ammonium containing repeat units, and if desired may contain other cationic groups such as imidazolyl, pyridinyl, and guanidino. The inventive cationic boronic acid polymers preferably contain both cationic repeat units and boronic acid repeat units. In some embodiments the inventive polymers may contain a repeat unit that contains a cationic group and a boronic acid group. The cationic polymers can be co-polymers that also contain any desired neutral or anionic repeat units, as further described herein, provided that the polymer retains a net cationic charge.

Pharmaceutical compositions comprising these polymers and methods of treatment using these polymers to treat metabolic disorders, such as Type-2 diabetes mellitus (T2DM), Type-1 diabetes mellitus (T1DM), prediabetes, hyperlipidemia, obesity, overweight, metabolic syndrome, non-alcoholic steatohepatitis, non-alcoholic fatty liver and polycystic ovary syndrome (PCOS) are also disclosed.

Exemplary polymers comprise a boronic acid moiety-containing repeat unit of Formula (I)-(III).

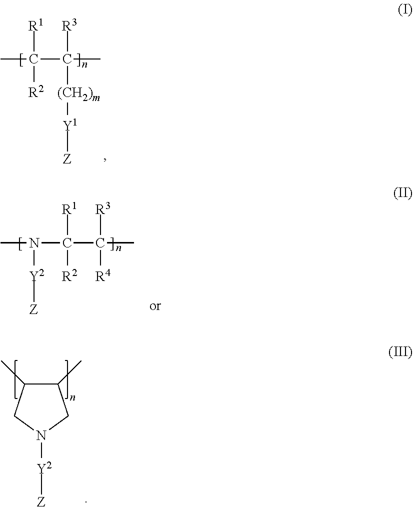

In Formulas (I) through (III):

R¹, R², R³ and R⁴ are independently hydrogen or substituted or unsubstituted alkyl;

Y¹ in each occurrence is independently a direct bond or -L¹-A¹-L²-A²-;

L¹ in each occurrence is —NR⁹—, —NC(O)— or —C(O)N—;

L² in each occurrence is absent, —NR⁹—, —O— or —S—;

Y² in each occurrence is independently a direct bond or -L³-A¹-L²-A²-;

L³ in each occurrence is —C(O)— or absent;

L² in each occurrence is absent, —NR⁹—, —O— or —S—;
A¹ and A² in each occurrence are independently absent or optionally substituted C₁-C₅ alkylene;
R⁹ in each occurrence is independently hydrogen or substituted or unsubstituted alkyl;

Z in each occurrence is

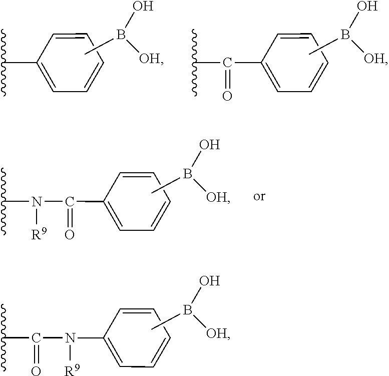

and preferably the —B(OH)₂ is at the 3- or 4-position of the ring;

n is an integer from 1 to 100,000 and

m is an integer from 0 to 4.

In some preferred aspects, the polymer contains a repeat unit of Formula (I).

Preferred repeat units of Formula (I) include repeat units of Formulas (Ia)-(If):

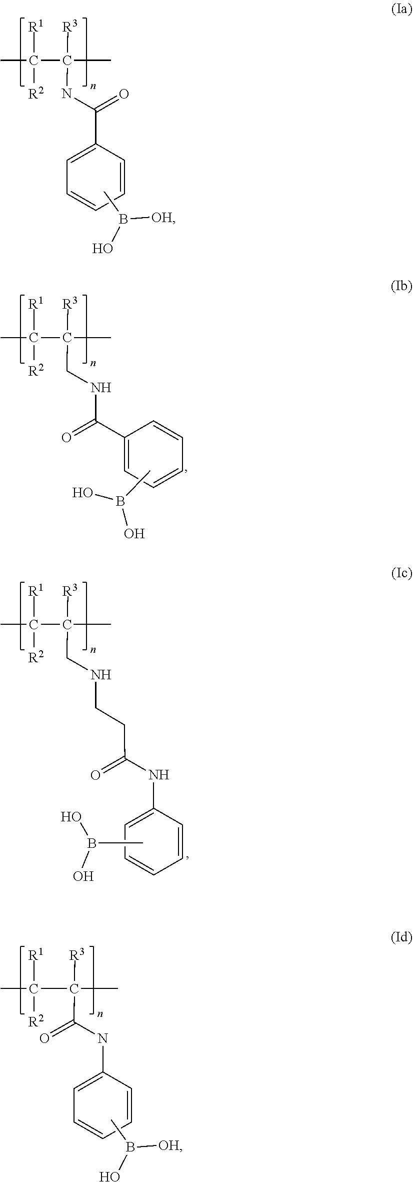

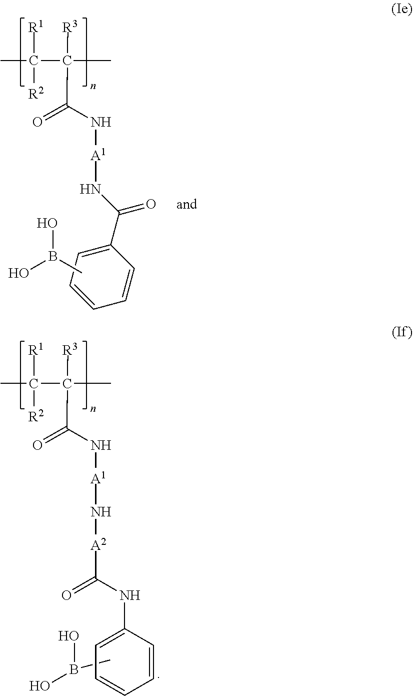

In Formulas (Ia) through (If), R¹, R², R³, A¹, A² and n are as described in Formula (I) and preferably the —B(OH)₂ is at the 3- or 4-position of the ring.

In some particular examples, the polymer includes a repeat unit of Formula (Ia), (Ib), (Ic), (Id), (Ie) or (If), wherein R¹, R² and R³ are each hydrogen.

In other particular examples, the polymer includes a repeat unit of Formula (Ia), (Ib), (Ic), (Id), (Ie) or (If) wherein R¹ and R² are each hydrogen, and R³ is alkyl, and preferably R³ is methyl.

In other preferred aspects, the polymer contains a repeat unit of Formula (II). In some examples of polymers that contain a repeat unit of Formula (II), Y² is a direct bond and Z is

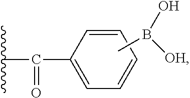

preferably the —B(OH)₂ is at the 3- or 4-position of the ring.

In other preferred aspects, the polymer contains a repeat unit of Formula (III). In some examples of polymers that contain a repeat unit of Formula (III), Y² is direct bond and Z is

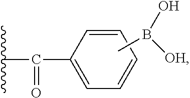

preferably the —B(OH)₂ is at the 3- or 4-position of the ring.

The polymers can be homopolymers. When homopolymers, the polymers contain a nitrogen-containing repeat unit (e.g. is a polyamine or polyamide) with pendant boronic acid moieties bonded to the polymer backbone directly or indirectly through the nitrogen atom of the repeat unit. Accordingly, the polymers typically contain secondary or tertiary amines, or quaternary ammonium if desired, to which the boronic acid moiety is bonded. The secondary or tertiary amines will be protonated at about pH 5-7, providing cationic polymers.

Preferably, the polymers are copolymers that contain a repeat unit of any one for Formulas (I)-(III) one or more other repeat units. The other repeat units are preferably cationic (e.g., a nitrogen-containing repeat unit), but can be neutral or anionic, provided that the polymer retains an overall cationic charge.

Preferred nitrogen-containing repeat units that can be modified to include pendant boronic acid moieties include poly(allylamine) (PAAn), poly(diallylamine) (PDAAn), poly(ethyleneimine) (PEI) and poly(methacrylamidopropylamine) (PMAPAn).

In the polymers disclosed herein, at least about 5% of the repeating chemical units contain a pendant boronic acid group, e.g., a repeat unit of any one of Formulas (I)-(III). In some instances, substantially all of the chemical repeat units in the polymer contain a pendant boronic acid group. Preferably, about 5% to about 50%, about 5% to about 40%, about 5% to about 30%, about 5% to about 20%, or about 5% to about 15% of the repeating chemical units contain a pendant boronic acid group.

Suitable nitrogen-containing repeat units for inclusion in the polymers are well-known in the art, and include for example, polyvinylamine, poly-N-alkylvinylamine, polyacrylamide, polyalkylacrylamides (e.g. polymethacrylamides), poly-N-alkylacrylamides, polyalkyl-N-alkylacrylamides, polyallylamine, poly-N-alkylallylamine, polydiallylamine, poly-N-alkyldiallylamine, polyethylenimine, polyaminostyrene, polyvinylimidazole, polyvinylpyridine, and the like.

Amine-containing repeat units are cationic when the amino nitrogen is protonated. If desired the cationic character can be altered using known methods, such as, by converting amines into guanidino, biguanide, aromatics such as imidazolyl and pyridinyl, quaternary ammoniums, or by introducing additional amino groups e.g., by alkylating an amine with an alkylamino or alkylammonium group.

Polyamines typically are highly charged at duodenal pH (about pH 5-6), however, due to a high density of protonated amine sites in close proximity, they deprotonate to a small extent as they pass from the stomach (pH˜2) to the duodenum (pH˜5) following ingestion. Even a small amount of neutralization effectively lowers the polymer charge density and causes these polymer chains become more coiled, compact, and less well hydrated as pH is increased. Without wishing to be bound by theory, this pH responsiveness is believed to contribute to preferential complexing of the mucus in the duodenum over the stomach. Other polymers of this invention which are capable of responding to the pH increase of the duodenum contain cationic repeat units (e.g., repeat units with protonated amines) that have inductive or structural features resulting in a lower pKa value than that of a standard protonated aliphatic amine. The lower pKa of these protonated polymers results in a greater sensitivity to the pH increase coincident with transit from the stomach to the duodenum. These types of polymers can therefore be targeted to interact with the loose mucus of the proximal duodenum, and include polyamines substituted with polar groups, such as hydroxyl groups less than three carbon atoms away from the protonated amine. In some embodiments, the polymers include amines that are modified to have a lower pKa than the unmodified amines. For example, the cationic polymers can have pKa values less than 9.0, more preferably a pKa less than 8.0, and most preferably a pKa less than or equal to 7.0.

Suitable boronic acid containing repeat units for inclusion in the polymers described herein include, but not limited to, for example, repeat units of Formulas (I), (Ia)-(If), (II) and (III), including the following:

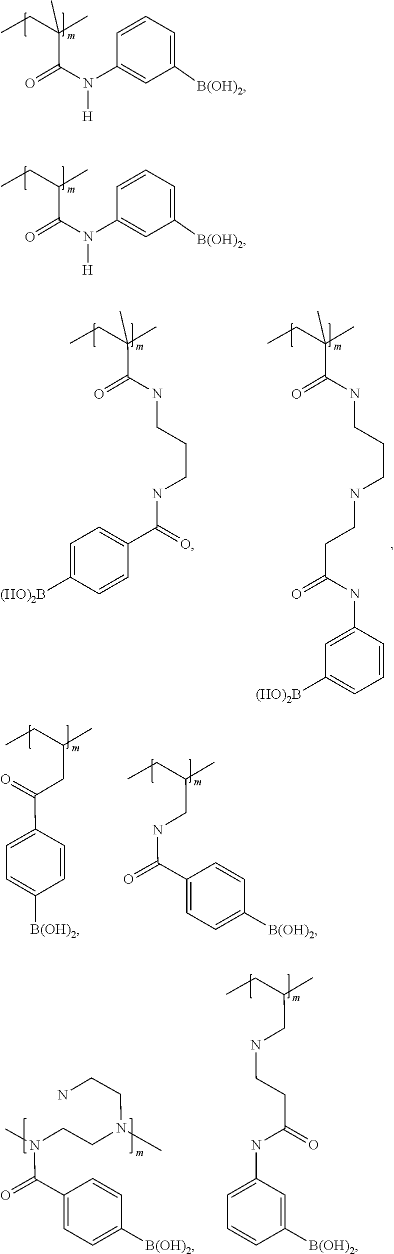

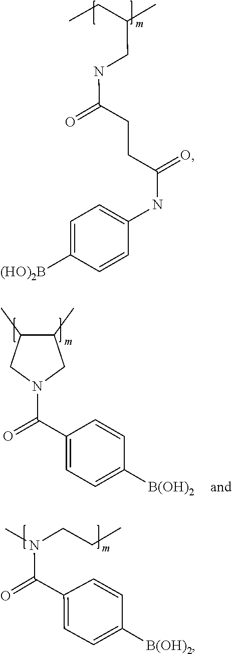

where “m” represents an integer from 1 to 100,000.

Suitable nitrogen-containing cationic monomeric repeat units include, but not limited to, for example, the following:

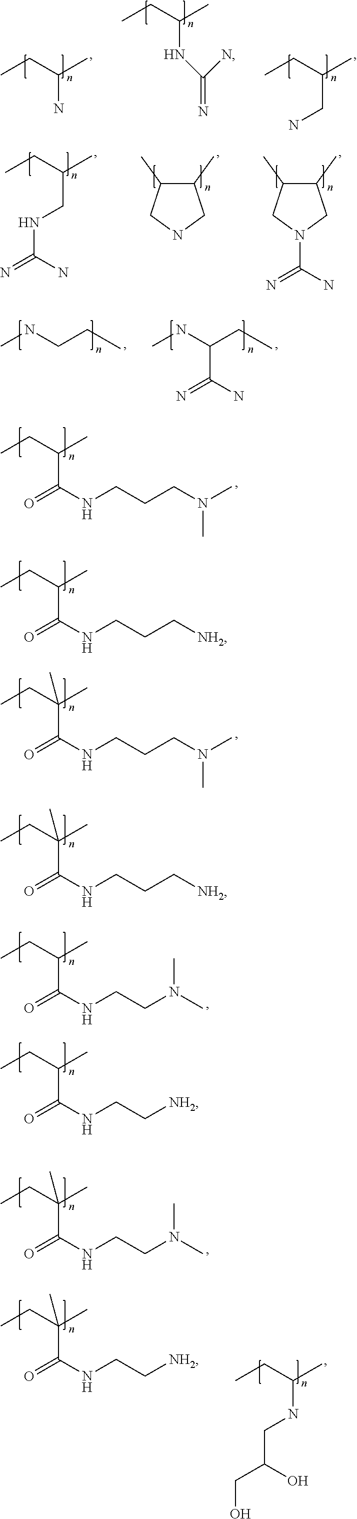

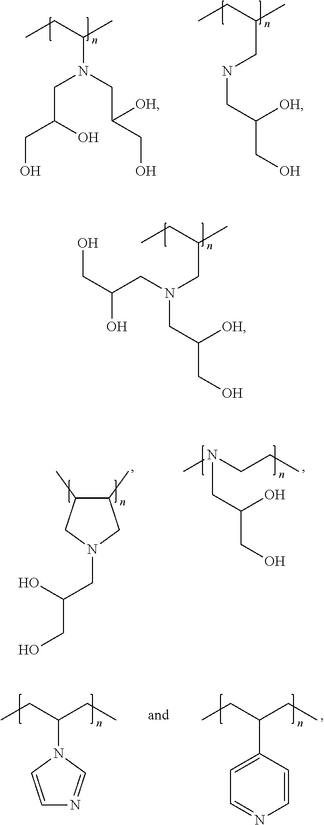

where “n” represents an integer from 1 to 100,000.

If desired, the cationic polymers that contain pendant boronic acid groups can also include a hydrophobic group, e.g., a pendent hydrophobic group. As used herein hydrophobic groups are moieties that are more soluble in octanol than water (as a separate chemical entity). For example, an octyl substituent is a hydrophobic group because octane is more soluble in octanol than in water. Suitable hydrophobic groups are C6 or greater linear, branched or cyclic hydrocarbons that can be substituted, for example with one or more hydroxy, halo, and/or aryl (e.g., benzyl) groups.

Additional repeat units that can be included in the polymers described herein, when desired, the polymer can include neutral or anionic repeat units, such as, polyacrylates, polyalkylene glycols, polystyrene, polyvinyl alcohols, polyvinylphosphates, polyvinylsulfates, and the like.

Particular examples of polymers of this disclosure include

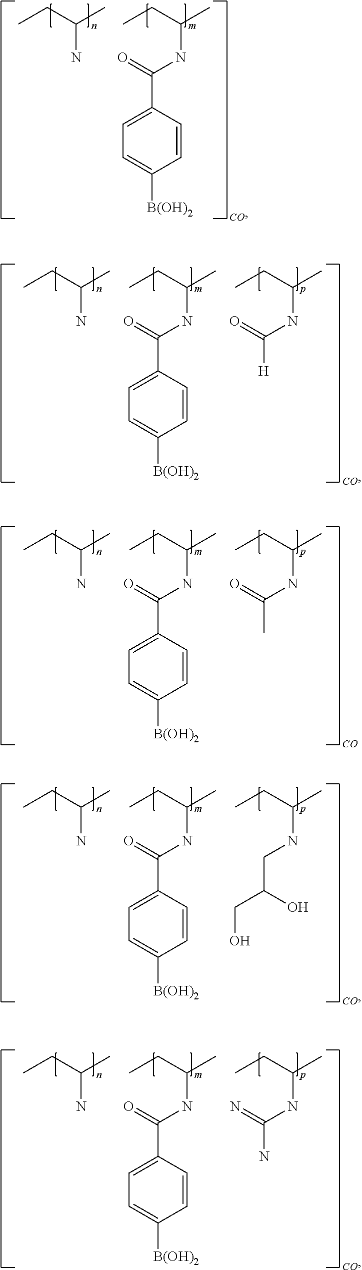

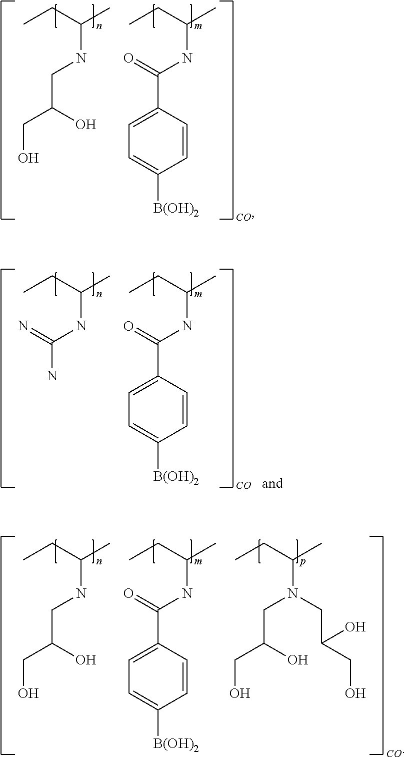

In some embodiments, polymers of the follow structures are excluded from the polymers of the compositions of the invention

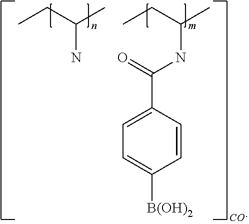

When the polymer is

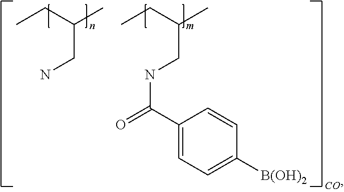

the polymer does not have a molecular weight that is greater than 100,000 or 75,000 or 50,000; or polymers of the structure

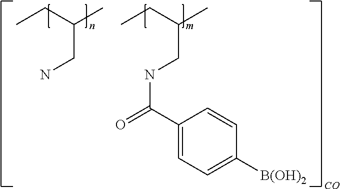

are excluded from the invention.

Additional, particular examples of polymers of this disclosure include

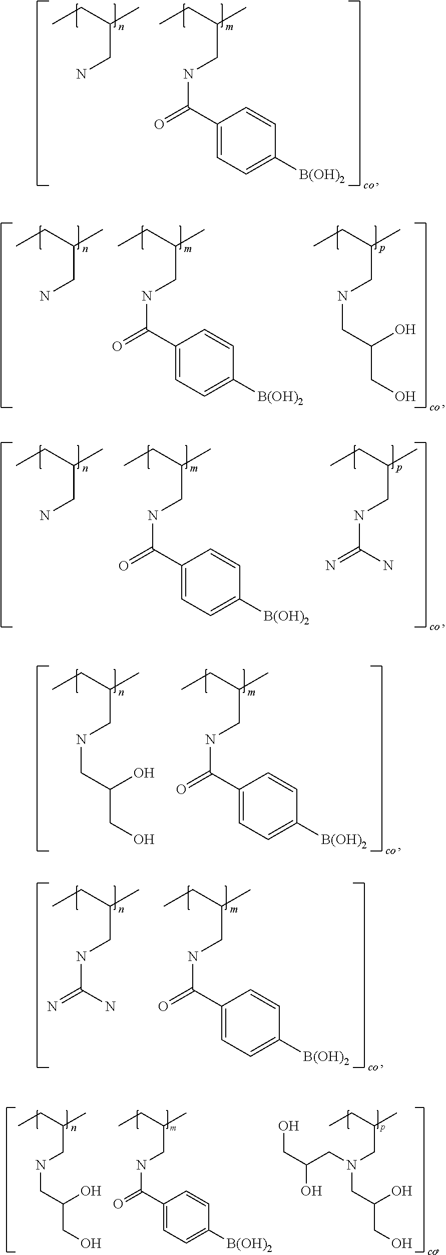

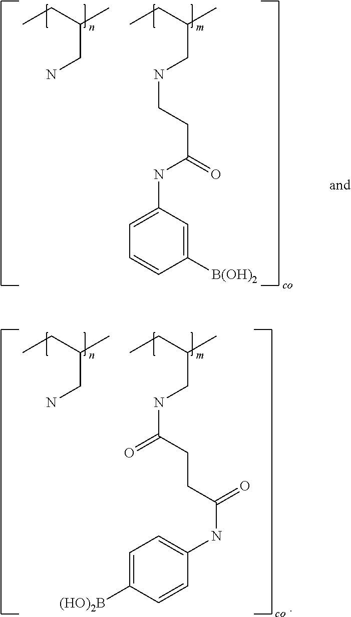

Additional particular examples of polymers of this disclosure include

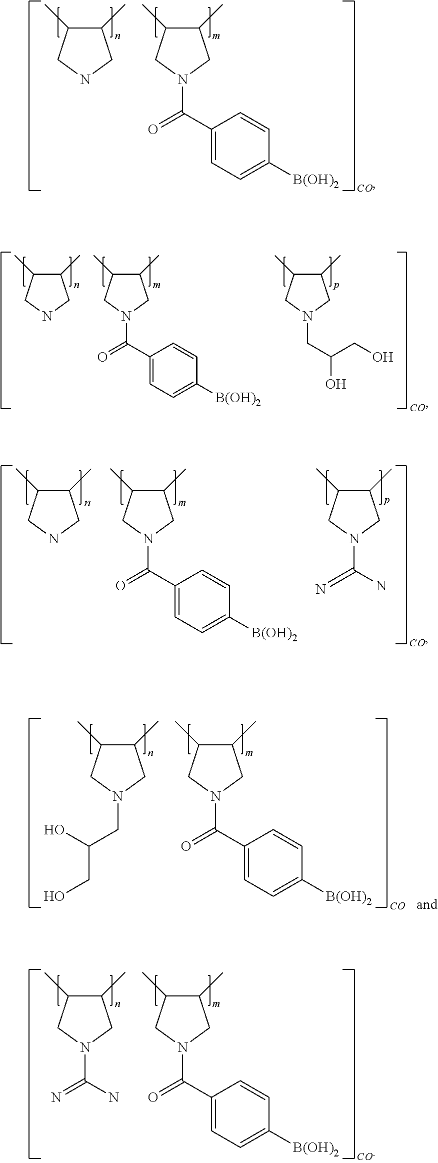

Additional particular examples of polymers of this disclosure include

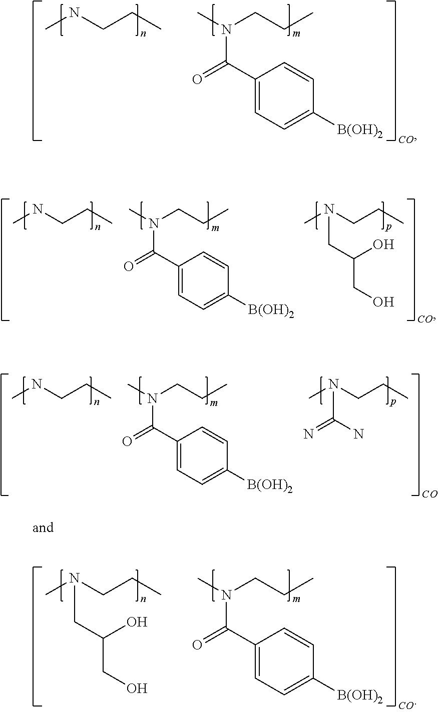

Additional particular examples of polymers of this disclosure include

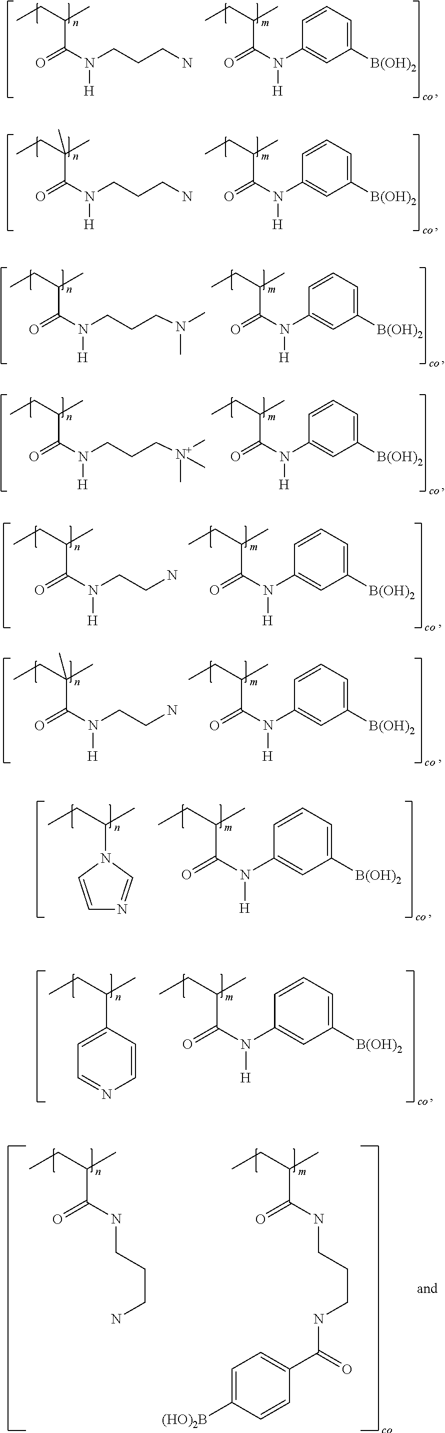

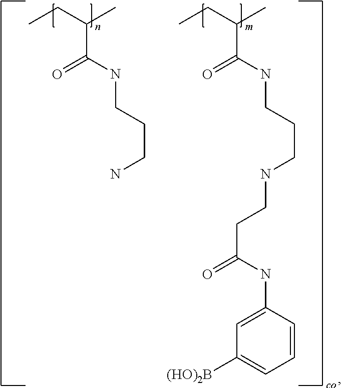

Copolymers of the present invention can exist in a variety of forms. Suitable forms include block copolymers, graft copolymers, comb copolymers, star copolymers, dendrimers, hyperbranched copolymers, random copolymers, gradient block copolymers, and alternating copolymers.

This disclosure also relates to cationic polymers that contain pendant hydrophobic groups, and to the use of such polymers for treating metabolic disease as disclosed herein.

Preferably, the polymers disclosed herein are of sufficient size so that the polymers are substantially not absorbed when administered orally to a subject, such as a human. The threshold molecular weight above which polymers are not absorbed from the GI tract into the systemic circulation is dependent on the specific polymer and conditions in the GI tract and other factors, but it is generally recognized that polymers of greater than 1,000 Da are not substantially absorbed from the GI tract into the systemic circulation. Accordingly, the compositions of this invention that are substantially not absorbed from the GI tract are substantially free of polymer chains smaller than 1,000 Da, and preferably 5,000 Da or more preferably 10,000 Da and have average molecular weights (M_{w}) of at least about 10,000 Da and preferably in the range of 20,000 to 250,000 Da or greater. The polymers of the invention can contain a distribution of polymer chain lengths, and may have a polydispersity index (PDI) in the 1.5-4.0 range, but they contain substantially no material under 1,000 Da, preferably they contain no material under 5,000 Da or more preferably under 10,000 Da.

The inventive polymers are soluble and are preferably not cross-linked. In some embodiments the polymers may be lightly cross-linked but remain soluble and do not form an extended network or gel.

Also included in the present invention are pharmaceutically acceptable salts of the disclosed polymers. For example, polymers which have acid functional groups can also be present in the anionic, or conjugate base, form, in combination with a cation. Suitable cations include alkaline earth metal ions, such as sodium and potassium ions, alkaline earth ions, such as calcium and magnesium ions, and unsubstituted and substituted (primary, secondary, tertiary and quaternary) ammonium ions. Polymers which have basic groups such as amines can also be protonated and have a pharmaceutically acceptable counter anion, such as halides (Cl⁻ and Br⁻), CH₃OSO₃⁻, HSO₄⁻, SO₄⁻, HCO³⁻, CO₃²⁻, nitrate, hydroxide, persulfate, sulfite, acetate, formate, sulfate, phosphate, lactate, succinate, propionate, oxalate, butyrate, ascorbate, citrate, dihydrogen citrate, tartrate, taurocholate, glycocholate, cholate, hydrogen citrate, maleate, benzoate, folate, an amino acid derivative, a nucleotide, a lipid, or a phospholipid. Similarly, ammonium groups comprise a pharmaceutically acceptable counteranion. Boronic acid groups can react with anions such as sodium or potassium hydroxide, alkoxide or carboxylate to form a salt such as —B—(OH)₃Na⁺, —B—(OH)₃K⁺, —B—(OH)₂(OCH₃)Na⁺, —B—(OH)₂(OCH₃)K⁺, —B—(OH)₂(OCOCH₃)Na⁺, —B—(OH)₂(OCOCH₃)K⁺, and the like.

The polymers disclosed herein are typically provided as a mixture of polymer chains with some variability in chain length. This distribution of polymer chain lengths can be measured using size exclusion chromatography (SEC) and a detector capable of measuring polymer molar mass such a multi-angle laser light scattering (MALLS). This method can also confirm the absence of short, low molecular weight polymer chains. It can also provide a polydispersity index (PDI), which is typically considered to be the ratio M_{w}/Mₙ, where M_{w} is the weight fraction-average molecular weight and Mₙ is the number average molecular weight.

PDI=M_{w}/Mₙ

Values of M_{w}, Mₙ and PDI can be obtained by SEC, preferable with a MALLS detector. For synthetic polymer materials made from standard free-radical processes, it is common to find PDI values greater than 2, and even greater than 3. In contrast, living free radical polymerization processes such as atom transfer radical polymerization (ATRP) or reversible addition fragmentation chain transfer (RAFT) are capable of producing materials with PDI less than 2, or even less than 1.5.

The polymers disclosed herein can be prepared using any suitable methods, such as by direct polymerization of one, two or more monomers or by polymer modification.

Polymerization can be accomplished using techniques known in the art of polymer synthesis (See, for example, Shalaby et al, ed., Water-Soluble Polymers, American Chemical Society, Washington, D.C. [1991]). Several cationic monomers are available as hydrochloride salts and can be polymerized by methods known in the art, for example, via a free radical addition process. In this case, the polymerization mixture includes a free-radical initiator. Suitable free-radical initiators include azobis(isobutyronitrile), azobis(4-cyanovaleric acid), 2,2′-azobis(2-amidinopropane)dihydrochloride, potassium persulfate, ammonium persulfate, and potassium hydrogen persulfate. Other suitable initiators include ionizing radiation and ultraviolet light. The free radical initiator is preferably present in the reaction mixture in an amount ranging from about 0.01 mole percent to about 5 mole percent relative to the monomer.

Polymer modification approach employs polyamines and copolymer approach employs acrylamide derivatives. “M” in the reaction schemes represent a group that includes a boronic acid moiety.

Polyamines can serve as mucus-interacting agents as well as starting materials for chemical modification with boronic acid groups. Exemplary polyamines include polyethyleneimine, hydroxyethylated polyethyleneimine, polyamidoamine (PAMAM) dendrimers, poly(allylamine) (PAAn) and its copolymers, poly(diallylamine) (PDAAn) and its copolymers, poly(vinylamine) and its copolymers, poly(vinylimidazole) and its copolymers, poly(vinylpyridine) and its copolymers, poly(vinylaniline) and its copolymers, amine containing acrylamide and methacrylamide copolymers, and the like. Preferred polyamines include poly(allylamine) (PAAn), poly(diallylamine) (PDAAn), poly(ethyleneimine) (PEI) and poly(methacrylamidopropylamine) (PMAPAn).

Polyamine derivatives can be obtained from the chemical modification of polyamines by amide-forming chemistry using EDC coupling (Scheme 1).

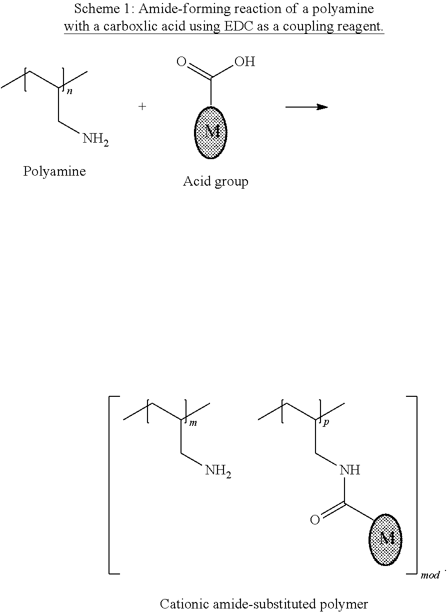

For example, a 1% wt/vol of desired polyamine can be prepared in deionized water with the pH adjusted to 5.0. Ethanol or other suitable organic is added to the polymer solution at 50% of initial polymer solution volume. The M-carboxylic acid to be coupled is placed into water at 25% of initial polymer solution volume to form a solution or slurry. The EDC coupling agent is dissolved in ethanol or other suitable solvent at 25% of initial polymer solution volume. The EDC solution is then mixed with the M-carboxylic acid solution or slurry. The combined EDC/M-carboxylic acid solution is added to the polymer solution dropwise by pipette or pressure equalizing addition funnel, over approximately 10 minutes. The reaction solutions contains polymer at about 0.5% wt/vol with about 62% vol water and about 38% vol ethanol or other suitable organic solvent. The reaction is stirred and pH is maintained at 5.0. With pH stabilized at 5.0, the reaction is allowed to stir at room temperature for about 18 hours. The polymer is precipitated with excess (3× volume) acetone.

The polyamine derivatives can be obtained from the chemical modification of polyamines by Michael addition reaction. The polyamine is the nucleophile and the acrylamides are the Michael acceptor (Scheme 2).

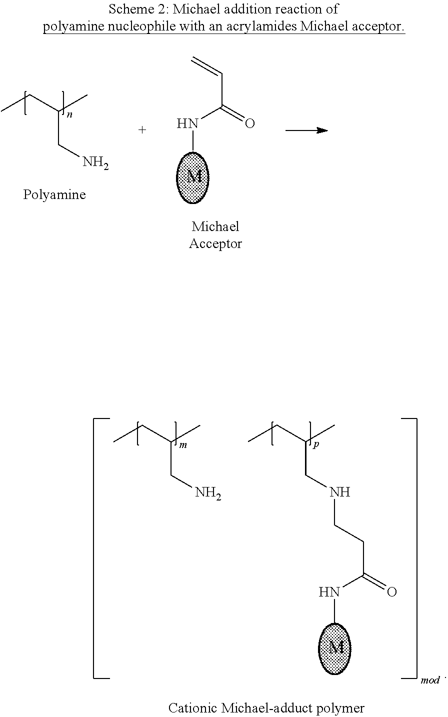

For example, a 1% wt/vol of desired polyamine is prepared in deionized water and the pH is adjusted to 8.5. This pH can be increased or lowered depending on level of modification desired. The desired M-acrylamide is dissolved in ethanol or other suitable solvent at 20% of initial polymer solution volume. The acrylamide solution is then added to the polymer solution to form a reaction mixture with the polymer at about 0.83% wt/vol with about 83% vol water and about 17% vol ethanol or other suitable solvent. The reaction mixture is heated to 70° C. and stirred for 48 hours. The polymer is precipitated with excess (3× volume) acetone.

The polyamine derivatives can be obtained from the chemical modification of polyamines by hydroxyalkylation using epoxide-opening chemistry (Scheme 3).

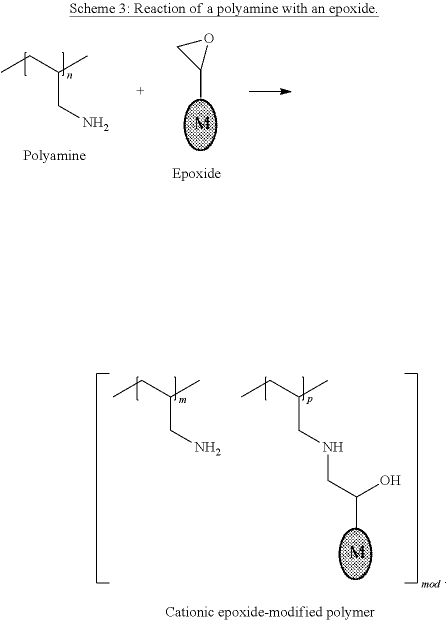

For example, a 2% wt/vol of desired polyamine is prepared in deionized water and the pH is adjusted to 6.0. This pH can be increased or lowered depending on level of modification desired. The desired M-epoxide is dissolved in water/ethanol (25%/75%) at 100% of initial polymer solution volume. This solution is then added to the polymer solution to prepare a reaction solution in which the polymer was about 1% wt/vol with about 62% vol water and about 38% vol ethanol. The reaction mixture was heated at 60° C. for 48 hr. If the epoxide is not fully in solution at 60° C., a small portion of additional ethanol may be added to aid in solubility. The polymer is precipitated with excess (3× volume) acetone.

Polyacrylamide derivatives can be obtained, for example, by the polymerization of 3-acrylamidopropylamine with a desired M-acrylate or M-acrylamide (Scheme 4).

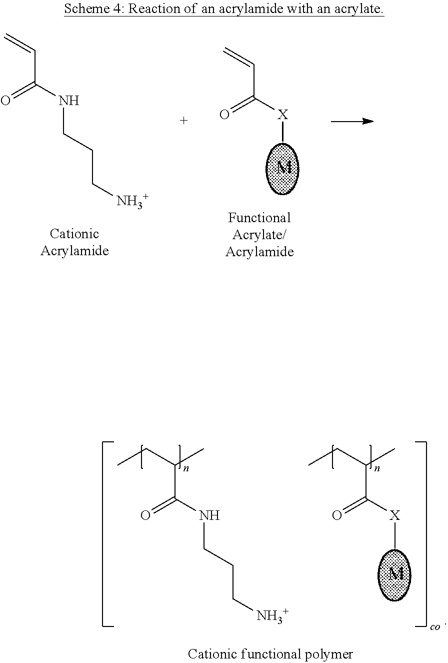

For example, a desired amount of the desired M-acrylate or M-acrylamide monomer is placed into a 30 ml glass vial equipped with magnetic stirring and an N₂ (g) inlet. The desired M-acrylate or M-acrylamide monomer is dissolved in dimethylformamide or other suitable water-miscible organic solvent. The desired amount of cationic, neutral or anionic co-monomer is then added. A small amount of water will likely be needed to fully dissolve the charged co-monomer in a binary solvent system. An appropriate amount of AIBN initiator is added. The co-monomer solution is N₂ (g) purged for at least ˜15 minutes. The reaction is then heated at 65° C. while under a blanket of N₂ (g). After several hours of heating, the copolymer solution or suspension is isolated by precipitation from acetone. The addition of some water and adjustment to lower pH will be needed for some of the polymers to facilitate precipitation in acetone. Finally, the product may be dissolved in deionized water, IPA/dry ice frozen and lyophilized.

As shown in the examples, the polymers described herein have improved mucus complexing activity in comparison to comparable cationic polymers, and can effectively condense mucus at the pH of the duodenum. The polymers bind tightly to mucus at the pH of the duodenum and once bound to mucus are resistant to removal by high concentrations of salts (e.g., 1M NaCl) or acidic conditions (e.g., 0.02M HCl). The resulting polymer-mucus complexes have dramatically different properties in comparison to free mucus. For example, as shown herein, the polymers reduce the diffusion coefficient of small particles in mucus by about two orders of magnitude (˜100×). When administered to a rat model of diabetes, the polymers effectively reduced blood glucose levels in an oral glucose tolerance test and showed dose responsive improvements in blood glucose levels. These results replicate the improvements in glucose homeostasis and reduced insulin resistance that are observed in patients that undergo “metabolic surgery” to bypass or exclude the duodenum from the digestive tract, but unlike surgery or invasive endoscopic procedures, the polymers of this invention are easily administered in a non-invasive manner. Without wishing to be bound by any particular theory or mechanism, these data demonstrate that the polymers are capable of complexing mucus in the duodenum to form an occlusive, non-absorbed luminal barrier when orally administered. The formation of the duodenal occlusive barrier is functionally similar or equivalent to bypassing, excluding, or ablating the duodenum by surgical or endoscopic procedures, and results in improved glucose homeostasis. (See, e.g., Koliaki, C. et al., BMC Endocr Disord. (2017) 17:50 DOI 10.1186/s12902-017-0202-6.)

Accordingly, in some aspects, the invention provides a method for applying a physical barrier to the gastrointestinal (GI) tract of a subject between the intestinal lining and the luminal contents. The method includes administering to the GI tract of the subject a therapeutic effective amount of a polymer described herein.

As used herein, the term “physical barrier” or “luminal barrier” refers to a complex of polymer and mucus that prevents or reduces chyme from contacting the mucosal epithelium located under the polymer-mucus complex in the intestinal tract. The physical barrier is created when the polymer combines in-situ with the anionic mucins contained within the mucus lining the wall of the intestines. The physical barrier can be substantially complete or partial. A substantially complete physical barrier extends to substantially cover an entire target area, such as the epithelial lining of the proximal duodenum. A partial physical barrier extends to cover a portion of a target area, such as a portion of the epithelial lining of the duodenum. For example, a partial barrier can cover at least about 1%, 5%, 10%, 20%, 25%, 30%, 40%, 50% or more of the epithelium at the target site.

The partial physical barrier can be discontinuous and spatially distributed, and may have varying degrees of permeability. For instance, physical barrier can be a semi-permeable complex of polymer and mucus or mucins on the luminal surface of the intestines, preferably in the duodenum.

In embodiments, the physical barrier or a formulation thereof is passed by natural digestive processes of the subject. In yet other embodiments, the physical barrier is removable or reversible by the ingestion of a liquid or solvent.

The polymers described herein bind tightly to the mucus and mucins to form a polymer-mucus complex that is resistant to disassociation (e.g., by high salt and low pH). Accordingly, once formed, the physical barrier will typically be present for a “retention period” or “residence time,” and is removed by the natural actions of the digestive system. Typical retention periods can range from about half an hour to about 7 days, including time period ranging from about 1 hour to about 3 hours, about 1 hour to about 5 hours, about 1 hour to about 24 hours, about 1 to about 3 days, and others.

The desired residence time can vary depending on the clinical application and can be adjusted based on the amount of polymer that is administered, and the frequency and interval between administrations. For instance, up to 50% of subjects with T2DM have gastroparesis, or delayed gastric emptying, that may require the mucoadhesive lining to remain in place for a longer time than a pre-diabetic or non-diabetic obese subject. Blood glucose levels spike often within the first two hours of eating a meal, most often within the first 60 minutes; thus, the lining should adhere for a minimum of 60 minutes in one embodiment. In another embodiment, in the case of pre-diabetic subjects who may not take medication prior to every meal, and thus may not comply with a treatment that would require to change their behavior, a longer lasting mucoadhesive lining may be required. In this application, the lining may adhere for a minimum of 6-8 hours with a maximum of 24 hours could be required. Residence time will also be influenced by the mucus layer at which the polymer develops the most affinity to. For instance, the superficial, loosely adherent layer sloughs off on the order of minutes to hours, whereas affinity to the deeper firmly adherent layer would lead to a longer lasting mucoadhesive coating. Overall, residence time can be tuned to various clinical and technical considerations in the embodiments outlined in this disclosure.

Polymers of the invention can form an occlusive barrier layer in the proximal intestine, specifically the duodenum. Preferably, the occlusive barrier is formed in the proximal duodenum or duodenal bulb. The polymers are therefore fully capable of forming a barrier layer immediately upon release from the stomach and entry into the proximal duodenum.

The polymers are administered orally in any suitable dosage form. A variety of dosage forms that are suitable for oral administration are well-known in the art and include, liquid formulations (e.g., solutions, suspensions, slurries, syrups), gels, ointments, powder, tablets, caplets, capsules and the like.

In one example, the polymers can be administered in a liquid form and are typically sufficiently stable and soluble in the stomach allowing immediate delivery to the duodenum in an active state without requiring further swelling, solubilization, or equilibration with the surrounding milieu. The polymers described herein are typically polyamines, which undergo some degree of deprotonation as they transition from the highly acidic stomach (pH˜2) to the duodenum (pH˜5) which targets the complexing activity of the polymers to the duodenum. However, the polymers may also form a barrier layer in the stomach.

In other examples, the polymer is administered in a solid form capable of being hydrated in the stomach. The solid form can be formulated to provide slow dissolution, which can protect the polymers from gastric acidity, but resulting in the polymers entering the proximal duodenum in a fully active state. In other examples, the polymers are administered in the form of an enteric-coated tablet, caplet, capsule or other enteric-coated dosage form to protect the polymers from gastric acidity. In such examples, the enteric coating is formulated to dissolve or degrade as soon as possible after or during passage through the pyloric valve (when the pH increases from pH˜2) permitting the immediate release of the polymers. Such dosage forms can include a superdisintegrant to facilitate immediate release of the polymers at the desired site in the intestinal tract, such as the proximal duodenum. Suitable superdisintegrant excipients are well-known in the art. (See, e.g., Mohanachandran, P. S. et al, Superdisintegrants: An Overview, Int. J. Pharma. Sci. Review and Research, (2011) 6:1 pp 105-109.) For example, enteric capsules have been described in the literature that are capable of targeting delivery to the duodenum. (See, e.g., Reix N. et al. Intl J Pharm (2012) 422:1-2 pp. 338-340.)

The polymers of the invention can quickly dissolve after oral administration in the stomach, in the duodenum or on other mucosal surfaces.

In some aspects, the pharmaceutical formulations of the polymers of this invention may optionally include a calcium salt, such as calcium chloride or calcium citrate. It is believed that the physical barrier formation can be accelerated in the presence of a calcium salt.

If desired, the polymers can be administered to the gastrointestinal tract of the subject via an endoscope, a nasal feeding tube, an oral feeding tube, or similar device. The polymer can also be sprayed onto the mucosa at the desired site of action, for example, the spraying can be done endoscopically.

For therapeutic purposes, a “therapeutically effective amount” of the polymer is administered. A therapeutically effective amount, as used herein is an amount sufficient to affect the desired response under the conditions of administration, including clinical response. The therapeutically effective amount may be sufficient, for example, to improve glucose homeostasis, to reduce insulin resistance, to cause weight loss, and/or to improve other signs and/or symptoms of T1DM, T2DM or other metabolic disorders such as hyperlipidemia, non-alcoholic steatohepatitis, non-alcoholic fatty liver and other conditions such as obesity and overweight. For example, a therapeutically effective amount can be an amount sufficient to lower blood glucose levels and/or reduce HbA1C.

The precise amount that is administered will depend on a number of well-known considerations, including the age, weight, gender, particular condition to be treated and its severity, sensitivity to drugs, and overall health of the subject. A skilled clinician can determine appropriate amounts to administer based on these and other considerations. Typically, 1 to 5 tablets/capsules are administered per dose, each of size 0 or 0E or 00 or 00E or 000. A dose can be administered one, two, three or four times per day. The dose timing will be based on the underlying indication. Preferably, a dose is administered at least 5, 10, 15, 30 or 60 min and not more than 12 hrs before a meal for the treatment of metabolic conditions. For other indications, dosing right before or along with food may be preferred.

As used herein and as well understood in the art, “treatment” is an approach for obtaining beneficial or desired results, including clinical results. Beneficial or desired clinical results may include, but are not limited to, alleviation or amelioration of one or more symptoms or conditions, diminution of extent of disease or affliction, a stabilized (i.e., not worsening) state of disease or affliction, preventing spread of disease or affliction, delay or slowing of disease or affliction progression, amelioration or palliation of the disease or affliction state and remission (whether partial or total), whether detectable or undetectable. “Treatment” can also mean prolonging survival as compared to expected survival if not receiving treatment.

This disclosure relates to methods of treating metabolic disease by administering a therapeutically effective amount of a polymer disclosed herein to a subject in need thereof. Metabolic diseases that can be treated using the method include, for example, glucose intolerance, T1DM, T2DM, prediabetes, hyperlipidemia, obesity, overweight, obesity, dyslipidemia, hypertension, hyperglycemia, impaired glucose tolerance, insulin resistance, metabolic syndrome, non-alcoholic steatohepatitis (NASH), non-alcoholic fatty liver disease (NAFLD) and polycystic ovary syndrome (PCOS).

This disclosure also relates to methods of treating gastrointestinal disorders by administering a therapeutically effective amount of a polymer disclosed herein to a subject in need thereof. Gastrointestinal disorders that can be treated using the method include, for example, celiac disease, irritable bowel syndrome, inflammatory bowel disease, colitis, Clostridium difficile, endotoxemia, diarrhea and constipation.

The methods of the invention are also useful in addressing leaky gut syndrome and associated conditions. Leaky gut syndrome is a term of art that describes a condition in which there is increased intestinal permeability due to alteration/damage to the tight epithelial junctions which results in a compromised epithelial barrier function. This impaired barrier acts as a conduit for intraluminal macromolecules and antigens to permeate through the gut wall triggering inflammatory, immunological reactions that result in various health conditions. For example, leaky gut syndrome has been implicated in IBS (irritable bowel syndrome). Certain proteins in foods can behave as antigens eliciting an immune response. For example, in Celiac disease, preventing gluten from coming into contact with the epithelium can reduce the immunological response. Leaky gut has also been implicated in other immunological conditions like Inflammatory Bowel Disease (Crohn's disease, Ulcerative Colitis). An enhanced intestinal barrier can reduce the absorption of endotoxins in the gastrointestinal tract. Some of these endotoxins are a result of normal bacterial metabolism/breakdown or due to bacterial overgrowth. This is particularly relevant in patients with impaired liver function, e.g., in liver cirrhosis, in whom the endotoxins are not metabolized (detoxified) by the liver resulting in impaired brain function (called hepatic encephalopathy). Thus the methods described herein can be used to enhance the barrier properties of the intestine and can treat or reduce the incidence of hepatic encephalopathy. Uremia is another condition associated with an impairment of intestinal barrier function that can be treated or reduced using the methods described herein. Similarly, the methods described herein can be used to treat or reduce the incidence of Chronic Kidney Disease (CKD), as clinical evidence has documented greater intestinal permeability in patients with advanced CKD.

The therapeutic methods can also provide benefit by reducing the clinical biomarkers associated with a variety of disorders, such as reducing systemic inflammation, oxidative stress and hyperuricaemia.

The disclosed polymers can be administered to the subjects in the form of a pharmaceutical composition that includes a pharmaceutically acceptable carrier, excipient, buffer or diluent.

For oral administration, the pharmaceutical compositions of the invention may be presented in dosage forms such as capsules, tablets, caplets, powders, granules, gels, suspensions, solutions or other suitable dosage form. Capsule may be gelatin, soft-gel or solid. Tablet, caplet and capsule formulations may further contain one or more adjuvants, binders, diluents, disintegrants, excipients, fillers, or lubricants, each of which are known in the art. Examples of such include carbohydrates such as lactose or sucrose, dibasic calcium phosphate anhydrous, corn starch, mannitol, xylitol, cellulose or derivatives thereof, microcrystalline cellulose, gelatin, stearates, silicon dioxide, talc, sodium starch glycolate, acacia, flavoring agents, preservatives, buffering agents, disintegrants, and colorants.

Orally administered compositions may contain one or more optional agents such as, for example, sweetening agents such as fructose, aspartame or saccharin; flavoring agents such as peppermint, oil of wintergreen, or cherry; coloring agents; and preservative agents, to provide a pharmaceutically palatable preparation. Suitable pharmaceutical formulations for oral administration and methods for preparing them are well-known in the art. See, e.g., Remington: The Science and Practice of Pharmacy, twentieth edition, 2000.

Pharmaceutical preparations that can be used orally include push-fit capsules made of a suitable material, such as gelatin, as well as soft, sealed capsules made of a suitable material, for example, gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration. Methods for encapsulating compositions (such as in a coating of hard gelatin or cyclodextran) are known in the art (Baker, et al., “Controlled Release of Biological Active Agents”, John Wiley and Sons, 1986).

The methods of the invention include a co-formulation of the polymeric composition comprising with probiotics. Probiotic formulations assist in building the beneficial probiotic bacteria in the intestinal tract. It is known in the art that probiotics have significant effects on the reduction of blood sugar, HbA1c, insulin levels and insulin resistance in subjects with diabetes. Suitable probiotics include, but not limited to Lactobacillus bifidobacteria, Saccharomyces boulardii, and Bacillus coagulans, Akkermansia muciniphila, Bifidobacterium spp, Escherichia spp. Methods to prepare formulations containing probiotics are well known in the art.

If desired, the therapeutic methods described herein can include co-administration of the polymeric compositions with one or more additional therapeutic agents. Therapeutic agents for co-administration in subjects with diabetes may include classes of drugs that are GLP-1 receptor agonists, DPP-4 inhibitors, SGLT-2 inhibitors, glucosidase inhibitors, insulin, metformin, sulfonylureas and thiazolidenediones.

In particular examples, the additional therapeutic agent is one or more agent indicated for the treatment of diabetes (type 1 and/or type 2), pre-diabetes, hyperglycemia, impaired glucose tolerance or insulin resistance. Such agents include biguanides (e.g., metformin), sulfonylureas (e.g., limepiride, gliclazide, gilpizide, glimepiride, tolbutamide, glibenclamide (glyburide), gliquidone, and glyclopyramide), meglinithinides (e.g., repaglinide and nateglinide); thiazolidindiones (e.g., pioglitazone and rosiglitazone), alpha-glucosidase inhibitors (e.g., acarbose and miglitol), dipeptidyl peptidase 4 (DPP4) inhibitors (e.g., vildagliptin, sitagliptin, saxagliptin, and linagliptin), GLP-1 analogues (e.g., exenatide, lixisenatide, dulaglutide, and liraglutide), sodium-glucose co-transporter 2 (SGLT2) inhibitors (e.g., dapagliflozin, ganagliflozin, and empagliflozin), amylin mimetics (e.g., pramlinitide), D2-dopamine agonists (e.g., bromocriptine), bile acid sequestrants (e.g., cholestyramine, colesevelam, colestilan, and colestimide), and insulin (e.g., human insulin, insulin glulisine, insulin lispro, insulin isophane human, insulin zinc suspension mixed bovine, insulin protamine zinc bovine, insulin isophane porcine, insulin isophane human, and the like).

The co-therapeutic methods can provide several advantages over monotherapy. For example, administering the polymer and an additional therapeutic agent can enhance the efficacy of and/or reduce the amount of additional therapeutic agent that is needed for the desired effect. Accordingly, undesired side effects of the additional therapeutic agent can be reduced or eliminated. Additionally, the polymers of the invention and the additional therapeutic can provide superior therapy in comparison to each agent as a monotherapy, and co-therapy can provide additive or synergistic effects.

The subject to be treated by the presently disclosed methods is typically a mammal and preferably a human subject. Suitable subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. Preferably, the subject is a human including, but not limited to, fetal, neonatal, infant, juvenile, and adult humans.

The terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

The term “about,” when referring to a value means ±20%, or ±10. Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

As used herein, the term ‘alkyl” refers to monovalent aliphatic hydrocarbon typically containing 1 to about 6 carbon atoms. An alkyl group can be straight chain, branched chain, monocyclic moiety or polycyclic moiety or combinations thereof. Suitable substituents for an alkyl group include aryl, —OH, halogen (—Br, —Cl, —I and —F), —O(R′), —O—CO—(R′), —CN, —NO₂, —COOH, —NH₂, —NH(R′), —N(R′)₂, —COO(R′), —CONH₂, —CONH(R′), —CON(R′)₂, —S(O)R′, —S(O)₂R′, —SH and —S(R′). Each R′ is independently an alkyl group or an aryl group. A substituted alkyl group can have more than one substituent. Examples of alkyl groups include methyl, ethyl, propyl, isopropyl, butyl, iso-butyl, sec-butyl, tert-butyl, pentyl, hexyl, cyclopropyl, cyclobutyl, cyclopentyl, cyclohexyl, norbornyl, and the like.

As used herein, the term ‘alkylene” refers to —(CH₂)ₓ—, that may be optionally substituted, where x is an integer between 1 to 5. Preferably, x is between 1 to 3, more preferably x is 1 or 2. Suitable substituents for an alkylene group are identical to those for alkyl groups.

As used herein, the term “alkoxy” refers to a group of formula —O-alkyl. Example of alkoxy groups include methoxy, ethoxy, propoxy (e.g., n-propoxy and isopropoxy), tert-butoxy, and the like.

As used herein, the term “aryl,” refer to stable aromatic monocyclic ring system having 3-7 ring atoms, of which all the ring atoms are carbon, and which may be substituted or unsubstituted. Aryl substituents include, —OH, halogen (—Br, —Cl, —I and —F), —O(R′), —O—CO—(R′), —CN, —NO₂, —COOH, —NH₂, —NH(R′), —N(R′)₂, —COO(R′), —CONH₂, —CONH(R′), —CON(R′)₂, —S(O)R′, —S(O)₂R′, —SH and —S(R′). Each R′ is independently an alkyl group.

As used herein, the term “aryloxy” refers to a group of formula —O-aryl. The aryl group may optionally substituted.

As used herein, the term “hydroxy” refers to a group of formula —OH.

As used herein, “halo” or “halogen” refers to F, Cl, Br, or I.

As used herein, the phrase “optionally substituted” means unsubstituted or substituted. As used herein, the term “substituted” means that a hydrogen atom is removed and replaced by a substituent. It is to be understood that substitution at a given atom is limited by valency.

The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLES

### I. Synthesis

### Example 1. Synthesis of Poly(Allylamine Hydrochloride)/PAAn-HCl

26.28 ml of allylamine was placed into a 250 ml, 2 neck round bottom flask place equipped with magnetic stirring and N₂(g) inlet. The temperature was brought to <5° C. with an ice bath. 28.74 ml of 37 wt % HCl was placed into a pressure equalizing funnel. HCl was slowly added over 90 minutes so as to not let the temperature raise above 35° C. while under a blanket of N₂(g). 11.0 ml of deionized water was added to make a 50 wt % solution of allylamine hydrochloride. A nominal amount of 37 wt % HCl was added to bring the solution pH down to 2.60. 328 mg (1 wt %) of V50 initiator was then added as a solid. The monomer solution was N₂(g) purged for ˜30 minutes. The reaction was then heated at 60° C. for 24 hr while under a blanket of N₂(g). The viscous reaction mixture was placed into MWCO: 6 k to 8 k cellulose dialysis membrane and dialyzed over 2 to 3 days with several water changes. The dialyzed solution was passed through 1 μm filter, IPA/dry ice frozen and lyophilized. A yield of 12.79 g was obtained as a white solid.

### Example 2. Synthesis of PAAn-4CPBA Amide (High Degree of Substitution)

2.05 g of poly(allylamine hydrochloride) and 200 ml of deionized water was placed into a 500 ml single neck round bottom flask equipped with magnetic stirring. 100 ml of ethanol was added. A slurry with 2.83 g of 4-carboxyphenylboronic acid (4-CPBA) and 50 ml of deionized water and a solution with 3.27 g of EDC-HCl and 50 ml of ethanol, denatured were made separately. The 4CPBA and EDC solutions were mixed together until mostly dissolved, ˜5 min. The “4CPBA-EDC” solution was added to the polymer solution over ˜5 min. The reaction was maintained at pH 5 with small amounts of saturated bicarbonate solution until stabilized, ˜30 min to 2 hr depending on scale. The reaction was proceeded for 18 hr while stabilized at pH 5. The contents of the reaction were precipitated from excess acetone, two times, followed by MWCO: 3.5 k cellulose membrane dialysis over 2 to 3 days with several water changes. The dialyzed solution was passed through 1 μm filter, IPA/dry ice frozen and lyophilized. A yield of 2.52 g was obtained as a white solid.

### Example 3. Synthesis of PAAn-4CPBA Amide (Low Degree of Substitution)

2.08 g of poly(allylamine hydrochloride) and 250 ml of deionized water was placed into a 500 ml single neck round bottom flask equipped with magnetic stirring. 50 ml of ethanol was added. A slurry with 1.41 g of 4-carboxyphenylboronic acid (4-CPBA) and 50 ml of ethanol, denatured and a solution with 1.59 g of EDC-HCl and 50 ml of ethanol, denatured were made separately. The 4CPBA and EDC solutions were mixed together until mostly dissolved, ˜5 min. The “4CPBA-EDC” solution was added to the polymer solution over ˜5 min. The reaction was maintained at pH 5 with small amounts of saturated bicarbonate solution until stabilized, ˜30 min to 2 hr depending on scale. The reaction was proceeded for 18 hr while stabilized at pH 5. The contents of the reaction were precipitated from excess acetone, two times, followed by MWCO: 3.5 k cellulose membrane dialysis over 2 to 3 days with several water changes. The dialyzed solution was passed through 1 μm filter, IPA/dry ice frozen and lyophilized. A yield of 2.04 g was obtained as a white solid.

### Example 4. Synthesis of PAAn-4CPBA Amide (High Degree of Substitution) Modified with Glycidol

100 mg of poly(allylamine hydrochloride)-4CPBA (high degree of substitution) and 10 ml of deionized water was placed into a 30 ml glass vial equipped with magnetic stirring. The pH was adjusted to 8.0 with 1N NaOH. The reaction was heated to 60° C. 62.6 μl of glycidol was added and heated for 48 hr. The contents of the reaction were precipitated from excess acetone, two times, and taken-up in deionized water, IPA/dry ice frozen and lyophilized. A yield of 119 mg was obtained as a white solid.

### Example 5. Guanidinylation of PAAn-4CPBA Amide (High Degree of Substitution)

100 mg of poly(allylamine hydrochloride)-4CPBA (high DS) and 10 ml of deionized water was placed into a 30 ml glass vial equipped with magnetic stirring. 92.3 mg of pyrazole carboxamidine-HCl was added. The pH was adjusted to 8.0 with 1N NaOH. The reaction was heated to 45° C. for 48 hr. The contents of the reaction were precipitated from excess acetone, two times, and taken-up in deionized water, IPA/dry ice frozen and lyophilized. A yield of 100 mg was obtained as a white solid.

### Example 6. Guanidinylation of PAAn-4CPBA Amide (Low Degree of Substitution)

100 mg of poly(allylamine hydrochloride)-4CPBA (low DS) and 10 ml of deionized water was placed into a 30 ml glass vial equipped with magnetic stirring. 274 mg of pyrazolecarboxamidine-HCl was added. The pH was adjusted to 9.5 with 1N NaOH. The reaction was heated to 45° C. for 48 hr. The contents of the reaction were precipitated from excess acetone, two times, and taken-up in deionized water, IPA/dry ice frozen and lyophilized. A yield of 118 mg was obtained as a white solid.

### Example 7. Synthesis of PAAn-Benzyl Amide (High Degree of Substitution)

2.01 g of poly(allylamine hydrochloride) and 200 ml of deionized water was placed into a 1 L single neck round bottom flask equipped with magnetic stirring. The pH was adjusted from ˜3.6 to 5.2 with a small amount of 1N NaOH. A solution of 2.36 g of benzoic acid (Bz) and 100 ml of ethanol, denatured and a solution of 4.18 g of EDC-HCl and 100 ml of ethanol, denatured was separately made. The Bz and EDC solutions were mixed until mostly dissolved, ˜5 min. The “Bz-EDC” solution was added to the polymer solution over ˜5 min. The reaction was maintained at pH 5 with small amounts of saturated bicarbonate solution until stabilized, ˜30 min to 2 hr depending on the scale. The reaction proceeded for 18 hr while stabilized at pH 5. The contents of the reaction were precipitated from excess acetone, one time, followed by MWCO: 6 k to 8 k cellulose membrane dialysis over 2 to 3 days with several water changes. The dialyzed solution was passed through 1 μm filter, IPA/dry ice frozen and lyophilized. A yield of 1.82 g was obtained as a white solid.

### Example 8. Synthesis of PAAn-APBA (High Degree of Substitution)

250 mg of poly(allylamine hydrochloride) and 25 ml of deionized water was placed into a 100 ml two neck round bottom flask equipped with magnetic stirring. The pH was adjusted from ˜3.5 to ˜8.5 with a small amount of 1N NaOH. A solution with 128 mg of 3-acrylamidophenyl-boronic acid (APBA) and 5 ml of ethanol, denatured was separately made. The APBA solution was added to the polymer solution and heated for 48 hr at 70° C. The contents of the reaction were precipitated from excess acetone, one time, and solids taken-up in deionized water, IPA/dry ice frozen and lyophilized. A yield of 308 mg was obtained as an off-white solid.

### Example 9. Synthesis of PAAn-APBA (Low Degree of Substitution)

250 mg of poly(allylamine hydrochloride) and 25 ml of deionized water was placed into a 100 ml, two neck round bottom flask equipped with magnetic stirring. The pH was adjusted from ˜3.5 to ˜8.5 with a small amount of 1N NaOH. A solution with 52 mg of 3-acrylamidophenyl-boronic acid (APBA) and 5 ml of ethanol, denatured was separately made. The APBA solution was to the polymer solution and heated for 48 hr at 70° C. The contents of the reaction was precipitated from excess acetone, one time, and solids taken-up in deionized water, IPA/dry ice frozen and lyophilized. A yield of 218 mg was obtained as an off-white solid.

### Example 10. Synthesis of PAAn-C8 (Low Degree of Substitution)

250 mg of poly(allylamine hydrochloride) and 18 ml of deionized water was placed into a 100 ml, two neck round bottom flask equipped with magnetic stirring. The pH was adjusted from ˜3.5 to ˜6.0 with a small amount of 1N NaOH. A solution with 409 μl of 1,2-epoxyoctane (C8) and 12 ml of ethanol, denatured was separately made. The C8 solution was added to the polymer solution and heated for 24 hr at 60° C. The contents of the reaction were precipitated from excess acetone, two times, and solids taken-up in deionized water, IPA/dry ice frozen and lyophilized. A yield of 169 mg was obtained as an off-white solid.

### Example 11. Synthesis of Poly(Methacrylamidopropylamine-Hydrochloride)/PMAPAn-HCl

2.01 g of methacrylamidopropylamine-hydrochloride and 6 ml of deionized water was placed into a 30 ml glass vial equipped with magnetic stirring and N₂(g) inlet. The pH was adjusted to 2.3 with small amount of 5N NaOH. 20 mg (1 wt %) of “V50” initiator was added. The monomer solution was N₂(g) purged for ˜15 minutes. The reaction was then heated at 60° C. for 24 hr while under a blanket of N₂(g). The solids were isolated by one precipitation from acetone, taken-up in fresh water, IPA/dry ice frozen and lyophilized. A yield of 1.74 g was obtained as a white solid.

### Example 12. Synthesis of PMAPAn-4CPBA (High Degree of Substitution)

250 mg of PMAPAn-HCl and 25 ml of deionized water was placed into a 100 ml single neck round bottom flask equipped with magnetic stirring. The pH was adjusted from ˜4 to ˜5 with small amount of 1N NaOH. A slurry with 232 mg of 4-carboxyphenylboronic acid (4-CPBA) and 12.5 ml of deionized water and a solution with 268 mg of EDC-HCl and 12.5 ml of ethanol, denatured were made separately. The 4CPBA and EDC solutions were mixed until mostly dissolved, ˜2 min. The “4CPBA-EDC” solution was added to the polymer solution over ˜2 min. The reaction was maintained at pH 5 with small amounts of saturated bicarbonate solution until stabilized, ˜30 min to 2 hr depending on the scale. The reaction proceeded for 18 hr while stabilized at pH 5. The solids were isolated by precipitation from acetone two times, and taken-up in deionized water, IPA/dry ice frozen and lyophilized. A yield of 1.74 g was obtained as a white solid.

### Example 13. Synthesis of Poly(Vinylamine)/PVAn

25.0 ml of N-vinylformamide was placed into a solution of 125 ml of IPA and 42 ml of deionized water contained in a 500 ml three neck, round bottom flask equipped with magnetic stirring and N₂(g) inlet. 1.00 g (4 wt %) of “V50” initiator was added. The monomer solution was N₂(g) purged for ˜45 minutes. The reaction was then heated at 60° C. for 2 hr while under a blanket of N₂(g). The poly(N-vinylformamide) precipitated out at this specific cosolvent composition. The mother liquor was carefully decanted off so as to not lose any polymer and then replaced with an equivalent volume of deionized water (˜150 ml). 14.07 g of solid NaOH pellets were added and the hydrolysis reaction heated for 24 hr at 75° C. Finally, 1N HCl was added to bring the pH down below 13, and the crude PVA was purified by MWCO: 6 k to 8 k cellulose membrane dialysis over 2 to 3 days with several water changes, IPA/dry ice frozen and lyophilized. A yield of 9.4 g of tacky golden-colored solids in the free base form was obtained.

### Example 14. Synthesis of PVAn-4CPBA (High Degree of Substitution)

250 mg of PVA (free base) and 28 ml of deionized water was placed into a 100 ml single neck round bottom flask equipped with magnetic stirring. Once dissolved, the pH was adjusted from ˜12 to ˜5 with ˜2 ml of 1N HCl. A slurry with 770 mg of 4-carboxyphenylboronic acid (4-CPBA) and 5 ml of ethanol, denatured and a solution with 890 mg of EDC-HCl and 5 ml of ethanol, denatured were made separately. The 4CPBA and EDC solutions were mixed until mostly dissolved, ˜2 min. The “4CPBA-EDC” solution was added to the polymer solution over ˜2 min. The reaction at maintained at pH 5 with small amounts of saturated bicarbonate solution until stabilized, ˜30 min to 2 hr depending on the scale. The reaction proceeded for 18 hr while stabilized at pH 5. The solids were isolated by precipitation from acetone, two times, and taken-up in deionized water, IPA/dry ice frozen and lyophilized. A yield of 378 mg was obtained as off-white solids.

### Example 15. Synthesis of PVAn-4CPBA (Low Degree of Substitution)

250 mg of PVAn (free base) and 28 ml of deionized water was placed into a 100 ml, single neck round bottom flask equipped with magnetic stirring. Once dissolved, the pH was adjusted from ˜12 to ˜5 with ˜2 ml of 1N HCl. A slurry with 309 mg of 4-carboxyphenylboronic acid (4-CPBA) and 5 ml of ethanol, denatured and a solution with 357 mg of EDC-HCl and 5 ml of ethanol, denatured were made separately. The 4CPBA and EDC solutions were mixed until mostly dissolved, ˜2 min. The “4CPBA-EDC” solution was added to the polymer solution over ˜2 min. The reaction was maintained at pH 5 with small amounts of saturated bicarbonate solution until stabilized, ˜30 min to 2 hr depending on scale. The reaction proceeded for 18 hr while stabilized at pH 5. The solids isolated by precipitation from acetone two times, and taken-up in deionized water, IPA/dry ice frozen and lyophilized. A yield of 286 mg was obtained as an off-white solid.

### Example 16. Synthesis of Poly(HPMA-co-APBA), 92.5/7.5

500 mg of 2-hydroxypropyl methacrylamide (HPMA) and 3.14 ml of dimethylformamide was placed into a 30 ml glass vial equipped with magnetic stirring and N₂(g) inlet, stirred until dissolved. 54 mg of 3-acrylamidophenylboronic acid (APBA) and 5 mg of AIBN initiator were added, stirred until dissolved. The co-monomer solution was N₂(g) purged for ˜10 minutes. The reaction was then heated at 65° C. while under a blanket of N₂(g). After several hours of heating, the reaction precipitated copolymer. The reaction mixture was completely dissolved with the addition of ˜3 ml of deionized water, precipitated the copolymer with excess acetone, one time. The solids were isolated and dissolved with deionized water, IPA/dry ice frozen and lyophilized. A yield of 475 mg was obtained as a white solid.

### Example 17. Synthesis of Poly(HPMA-co-APBA), 85/15

500 mg of 2-hydroxypropyl methacrylamide (HPMA) and 3.50 ml of dimethylformamide was placed into a 30 ml glass vial equipped with magnetic stirring and N2(g) inlet, stirred until dissolved. 118 mg of 3-acrylamidophenylboronic acid (APBA) and 6 mg of AIBN initiator was added, stirred until dissolved. The co-monomer solution was N₂(g) purged for ˜10 minutes. The reaction was then heated at 65° C. while under a blanket of N₂(g). After several hours of heating, the reaction precipitated copolymer. The reaction mixture was completely dissolved with the addition of ˜3 ml of deionized water. The copolymer was precipitated with excess acetone, one time. The solids were isolated and dissolve with deionized water, IPA/dry ice frozen and lyophilized. A yield of 488 mg was obtained as a white solid.

### Example 18. Synthesis of Poly(HPMA-co-APBA), 70/30

500 mg of 2-hydroxypropyl methacrylamide (HPMA) and 4.45 ml of dimethylformamide was placed into a 30 ml glass vial equipped with magnetic stirring and N2(g) inlet, stirred until dissolved. 286 mg of 3-acrylamidophenylboronic acid (APBA) and 8 mg of AIBN initiator was added, stirred until dissolved. The co-monomer solution was N₂(g) purged for ˜10 minutes. The reaction was then heated at 65° C. while under a blanket of N₂(g). After several hours of heating, the reaction precipitated copolymer. This material was not water soluble and the addition of water doesn't completely dissolve the polymer.

### Example 19. Synthesis of Poly(MAPAn-co-APBA), 92.5/7.5

508 mg of methacrylamido-3-propylamine HCl (MAPAn), 3.08 ml of dimethyl formamide and 0.924 ml deionized water was placed into a 30 ml glass vial equipped with magnetic stirring and N₂(g) inlet, stirred until dissolved. Add 43 mg of 3-acrylamidophenylboronic acid (APBA) and 5 mg of AIBN initiator, stirred until dissolved. The co-monomer solution was N₂(g) purged for ˜15 minutes. The reaction was then heated for at 65° C. for ˜18 hr while under a blanket of N₂(g). The viscous copolymer solution was precipitated into excess acetone, one time. The solids were isolated, dissolved with deionized water, IPA/dry ice frozen and lyophilized. A yield of 245 mg was obtained as a white solid.

### Example 20. Synthesis of Poly(MAPAn-co-APBA), 85/15

500 mg of methacrylamido-3-propylamine HCl (MAPAn), 3.37 ml of dimethyl form-amide and 1.01 ml deionized water was placed into a 30 ml glass vial equipped with magnetic stirring and N₂(g) inlet, stirred until dissolved. 94 mg of 3-acrylamidophenylboronic acid (APBA) and 6 mg of AIBN initiator were added, stirred until dissolved. The co-monomer solution was N₂(g) purged for ˜15 minutes. The reaction was then heated for at 65° C. for ˜18 hr while under a blanket of N₂(g). The viscous copolymer solution was precipitated into excess acetone, one time. The solids were isolated, dissolved with deionized water, IPA/dry ice frozen and lyophilized. A yield of 357 mg was obtained as a white solid.

### Example 21. Synthesis of Poly(MAPAn-co-APBA), 70/30

505 mg of methacrylamido-3-propylamine HCl (MAPAn), 4.13 ml of dimethyl form-amide and 1.24 ml deionized water was placed into a 30 ml glass vial equipped with magnetic stirring and N₂(g) inlet, stirred until dissolved. 229 mg of 3-acrylamidophenylboronic acid (APBA) and 7 mg of AIBN initiator was added, stirred until dissolved. The co-monomer solution was N₂(g) purged for ˜15 minutes. The reaction was then heated for at 65° C. for ˜18 hr while under a blanket of N₂(g). The viscous copolymer solution was precipitated into excess acetone, one time. The solids were isolated, dissolved with deionized water, IPA/dry ice frozen and lyophilized. A yield of 400 mg was obtained as a white solid.

### Example 22. Synthesis of Poly(AMPS-co-APBA), 92.5/7.5

504 mg of 2-acrylamido-2methylpropane sulfonic acid (AMPS) and 3.04 ml of dimethylformamide was added into a 30 ml glass vial equipped with magnetic stirring and N₂(g) inlet, stirred until dissolved. 37 mg of 3-acrylamidophenylboronic acid (APBA) and 5 mg of AIBN initiator was added, stirred until dissolved. The co-monomer solution was N₂(g) purged for ˜15 minutes. The reaction was then heated at 65° C. for ˜18 hr while under a blanket of N₂(g). The viscous copolymer solution was precipitated into excess acetone, one time. The solids were isolated, dissolved with deionized water, IPA/dry ice frozen and lyophilized. A yield of 309 mg was obtained as a white solid.

### Example 23. Synthesis of Poly(AMPS-co-APBA), 85/15

508 mg of 2-acrylamido-2methylpropane sulfonic acid (AMPS) and 3.29 ml of dimethylformamide was placed into a 30 ml glass vial equipped with magnetic stirring and N₂(g) inlet, stirred until dissolved. 81 mg of 3-acrylamidophenylboronic acid (APBA) and 6 mg of AIBN initiator was added, stirred until dissolved. The co-monomer solution was N₂(g) purged for ˜15 minutes. The reaction was then heated at 65° C. for ˜18 hr while under a blanket of N₂(g). The viscous copolymer solution was precipitated into excess acetone, one time. The solids were isolated, dissolved with deionized water, IPA/dry ice frozen and lyophilized. A yield of 255 mg was obtained as a white solid.

### Example 24. Synthesis of Poly(AMPS-co-APBA), 70/30

500 mg of 2-acrylamido-2methylpropane sulfonic acid (AMPS) and 3.95 ml of dimethylformamide was placed into a 30 ml glass vial equipped with magnetic stirring and N₂(g) inlet, stirred until dissolved. 197 mg of 3-acrylamidophenylboronic acid (APBA) and 7 mg of AIBN initiator was added, stirred until dissolved. The co-monomer solution was N₂(g) purged for ˜15 minutes. The reaction was then heated at 65° C. for ˜18 hr while under a blanket of N₂(g). The viscous copolymer solution was precipitated into excess acetone, one time. The solids were isolated, dissolved with deionized water, IPA/dry ice frozen and lyophilized. A yield of 250 mg was obtained as a white solid.

### Example 25. Synthesis of Poly(VI-co-APBA), 80/20

254 mg of 3-acrylamidophenylboronic acid (APBA) and 3.02 ml of dimethylformamide was placed into a 30 ml glass vial equipped with magnetic stirring and N₂(g) inlet, stirred until dissolved. 482 μl of 1-vinylimidazole (VI) and 7 mg of AIBN initiator was added, stirred until dissolved. The co-monomer solution was N₂(g) purged for ˜15 minutes. The reaction was then heated at 65° C. while under a blanket of N₂(g). After several minutes of heating, the reaction precipitated copolymer. The reaction mixture was completely dissolved with the addition of ˜12 ml of deionized water and pH adjusted to ˜3.0 with 1N HCl. The copolymer was precipitated from excess acetone, two times, after first removing most of the water through rotary evaporation. The solids were isolated, dissolved with deionized water, IPA/dry ice frozen and lyophilized. A yield of 59 mg was obtained as a white solid.

### Example 26. Synthesis of Poly(VPy-co-APBA), 80/20

227 mg of 3-acrylamidophenylboronic acid (APBA) and 2.91 ml of dimethylformamide was placed into a 30 ml glass vial equipped with magnetic stirring and N₂(g) inlet, stirred until dissolved. 520 μl of 4-vinylpyridine (VPy) and 7 mg of AIBN initiator was added, stirred until dissolved. The co-monomer solution was N₂(g) purged for ˜15 minutes. The reaction was then heated at 70° C. while under a blanket of N₂(g). After several hours of heating, the reaction precipitated copolymer. The reaction mixture was completely dissolved with the addition of ˜12 ml of deionized water and pH adjustment to ˜3.0 with 1N HCl. The copolymer was precipitated from excess acetone one time, after first removing most of the water through rotary evaporation. The solids were isolated, dissolved with deionized water, IPA/dry ice frozen and lyophilized. A yield of 670 mg was obtained as a white solid.

### Example 27. Synthesis of Poly(APDMAn-co-APBA), 70/30

262 mg of 3-acrylamidophenylboronic acid (APBA) and 3.05 ml of dimethylformamide was placed into a 30 ml glass vial equipped with magnetic stirring and N₂(g) inlet, stirred until dissolved. 526 μl of acrylamidopropyl-3-dimethylamine (APDMAn) and 8 mg of AIBN initiator was added, stirred until dissolved. The co-monomer solution was N₂(g) purged for ˜15 minutes. The reaction was then heated at 65° C. while under a blanket of N₂(g). After several minutes of heating, the reaction precipitated copolymer. The reaction mixture was completely dissolved with the addition of ˜12 ml of deionized water and pH adjustment to ˜3.0 with 1N HCl. The copolymer was precipitated from excess acetone, two times, after first removing most of the water through rotary evaporation. The solids were isolated, dissolved with deionized water, IPA/dry ice frozen and lyophilized. A yield of 154 mg was obtained as a white solid.

### Example 28. Synthesis of Poly(APTAC-co-APBA), 70/30

199 mg of 3-acrylamidophenylboronic acid (APBA) and 1.95 ml of dimethylformamide was placed into a 30 ml glass vial equipped with magnetic stirring and N₂(g) inlet, stirred until dissolved. 675 μl of deionized water, 665 μl of 75% acrylamidopropyl trimethylammonium chloride (APTAC) and 7 mg of AIBN initiator was added, stirred until dissolved. The co-monomer solution was N₂(g) purged for ˜15 minutes. The reaction was then heated at 65° C. while under a blanket of N₂(g). After several minutes of heating, the reaction became highly viscous and semi-solid like. The reaction mixture was completely dissolved with just the addition of ˜4 ml of deionized water. The copolymer was precipitated from excess acetone, one time, after first removing most of the water through rotary evaporation. The solids were isolated, dissolved with deionized water, IPA/dry ice frozen and lyophilized. A yield of 583 mg was obtained as a white solid.

### Example 29. Synthesis of Poly(acrylamide-co-APBA), 70/30

253 mg of 3-acrylamidophenylboronic acid (APBA) and 2.83 ml of dimethylformamide was placed into a 30 ml glass vial equipped with magnetic stirring and N₂(g) inlet, stirred until dissolved. 243 mg of acrylamide and 6 mg of AIBN initiator was added, stirred until dissolved. The co-monomer solution was N₂(g) purged for ˜15 minutes. The reaction was then heated at 65° C. while under a blanket of N₂(g). After ˜18 hr of heating, the viscous reaction mixture was precipitated into excess acetone, one time. The solids isolated, dissolved with deionized water, IPA/dry ice frozen and lyophilized. A yield of 170 mg was obtained as a white solid.

### Example 30. Synthesis of Poly(Acrylic Acid-co-APBA), 70/30

249 mg of 3-acrylamidophenylboronic acid (APBA) and 2.83 ml of dimethylformamide was placed into a 30 ml glass vial equipped with magnetic stirring and N₂(g) inlet, stirred until dissolved. 224 μl of acrylic acid and 5 mg of AIBN initiator was added, stirred until dissolved. The co-monomer solution was N₂(g) purged for ˜15 minutes. The reaction was then heated at 65° C. while under a blanket of N₂(g). After ˜18 hr of heating, the viscous reaction mixture precipitated into excess acetone, one time. The solids isolated, dissolved with deionized water, IPA/dry ice frozen and lyophilized. A yield of 168 mg was obtained as a white solid.

### Example 31. Synthesis of Poly(HEAAm-co-APBA), 80/20

145 mg of 3-acrylamidophenylboronic acid (APBA) and 2.84 ml of dimethylformamide was placed into a 30 ml glass vial equipped with magnetic stirring and N₂(g) inlet, stirred until dissolved. 319 μl of 2-hydroxyethyl acrylamide (HEAAm) and 5 mg of AIBN initiator was added, stirred until dissolved. The co-monomer solution was N₂(g) purged for ˜15 minutes. The reaction was then heated at 65° C. while under a blanket of N₂(g). After ˜18 hr of heating, the viscous reaction mixture precipitated into excess acetone, one time. The solids were isolated, dissolved with deionized water, IPA/dry ice frozen and lyophilized. A yield of 483 mg was obtained as a white solid.

### Example 32. Synthesis of (PAAn-HCl) Modified with CPBA Materials

Poly(allylamine hydrochloride) was obtained from Nittobo Medical, Japan (PAAn-HCl, Cat #PAA-HCl-3L, 50.3% solution in water) and used as received. The material was qualified by 1H-NMR, TGA, and size exclusion chromatography (SEC-MALLS). 4-Carboxyphenylboronic acid was obtained from Chem-Impex International, Wood Dale, Ill. (CPBA, Cat #28086, 99.7%) and used as received. The material was qualified by 1H-NMR, FT-IR, and melting point. 1-(3-Dimethylaminopropyl)-3-ethylcarbodiimide hydrochloride was obtained from Chem-Impex International, Wood Dale, Ill. (EDC-HCl, Cat #00050, 99.8%) and used as received. The material was qualified by 1H-NMR, FT-IR, and melting point. 1-Hydroxybenzotriazole hydrate was obtained from Chem-Impex International, Wood Dale, Ill. (HOBt, Cat #24755, 99.8% (odb), 21.3% water) and used as received. The material was qualified by 1H-NMR, and FT-IR.

The reaction was carried out using 114 g PAAn-HCl in a 5-liter volume. On this basis, the reaction was performed at 2.3% polymer solids. The reaction was carried out at room temperature which was 22±3° C. Temperature monitoring of the reaction with a thermocouple indicated no exotherms or temperature excursions outside of this range during the reaction.

Synthesis:

The 50.3% PAAn solution (225.92 g, 1.215 amine equivalents) was placed in a 10-liter beaker with a magnetic stir bar and deionized water (4,333 ml). The resulting clear solution was magnetically stirred, and a pH electrode introduced. The pH was adjusted to 8.0 by dropwise addition of a 5N NaOH solution while stirring. Solid 99.7% CPBA powder (32.35 g, 0.194 mol) was added to the reaction mixture and the resulting suspension was stirred. After 20 minutes of stirring, the pH had dropped to 6.7 and some solid CPBA remained suspended in solution. Additional NaOH solution was added in portions with stirring to cause the complete dissolution of the CPBA suspension. The pH of the resulting clear solution was 7.5. Solid 78.7% HOBt hydrate powder (1.67 g, 0.010 mol) was then added to the clear reaction solution and dissolved after 20 minutes of stirring. Hydrochloric acid (4N) was added to the reaction mixture to lower the pH to 5.5. The reaction solution remained clear. Solid 99.8% EDC-HCl powder (41.06 g, 0.214 mol) dissolved in 100 ml of deionized water was then slowly poured into the reaction mixture with good stirring. The clear reaction mixture had a final volume of 5-liters and a pH of 5.5-5.7. This reaction mixture was stirred for 18 hours. Sodium chloride (125 g) was added to the reaction mixture and 1N HCl was added to bring the pH to 2.5. The reaction mixture was then filtered through a 0.2-micron PES filter device into a 5-liter screw-cap bottle and stored at 2-5° C.

Purification:

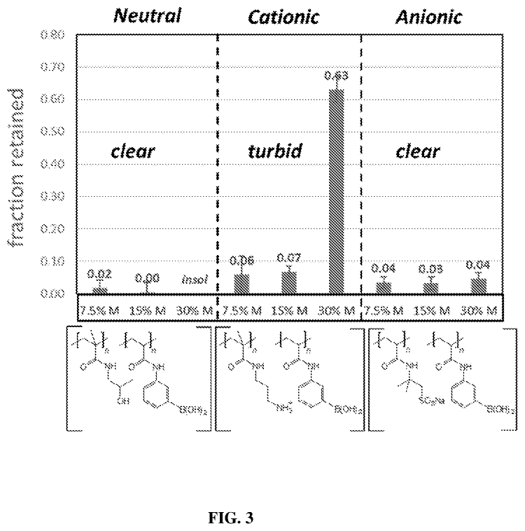

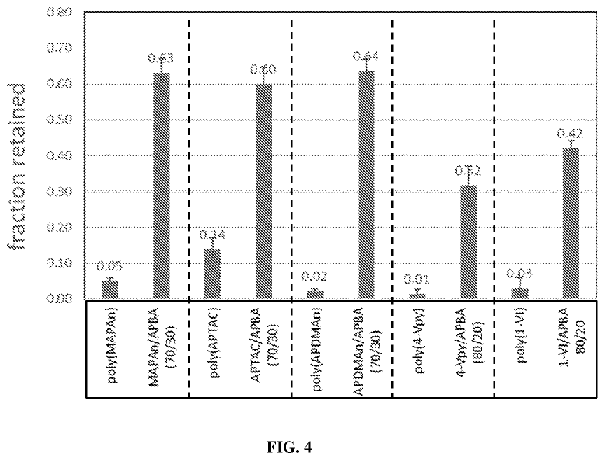

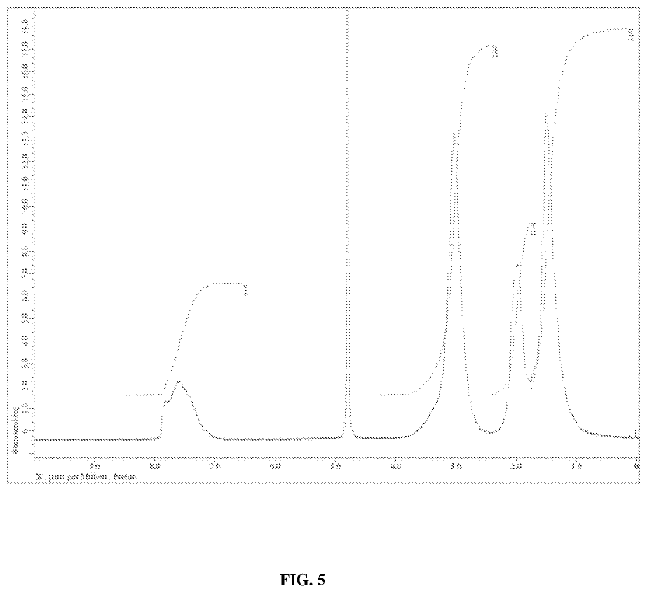

The filtered reaction mixture prepared above was subjected to purification by tangential-flow filtration (TFF) using two Centramate™ (Pall Corp.) 30 kDa MWCO TFF cassettes in series. The initial 5,000 ml reaction mixture was first concentrated by TFF to a working volume of 2,500 ml. This concentrated solution was then subjected to diafiltration by TFF with continuous replenishment using a deionized water solution containing 2.5% (wt/vol) sodium chloride acidified with concentrated HCl to a pH of 2.5. TFF was continued until 4 volumes (10-liters) of filtrate were collected. At that time, TFF was suspended and the working solution in the reservoir was brought to pH 7.9 by the addition of 1N NaOH. Desalting of this solution was then achieved by TFF with replenishment by deionized water until 6-liters of filtrate was collected and the conductivity of the filtrate was 100 microsiemens per centimeter (uS/cm). The purified polymer solution was recovered from the TFF device along with a small deionized water rinse (50-60 ml) of the device. The collected clear solution was found to have a weight of 2,357 g and a pH of 7.5. The purified polymer solution was stored at 2-5° C. Small samples (3×0.5 ml) of this polymer solution were placed in tared vials and lyophilized revealing a solid content of 4.91%. On this basis, the amount of purified polymer recovered was 116 g. One of the samples lyophilized for solids determination was dissolved in D₂O/DCl and examined by 1H-NMR (FIG. 5).

Isolation:

The purified polymer solution was pipetted into a set of 50 ml vials (20 ml solution per vial) and lyophilized on a shelf lyophilizer. The vials were sealed under dry nitrogen, providing the purified polymer product as a white solid.

### II. Mucin-Mixing Assay

When a water-soluble mucin glycoprotein solution is mixed with certain water-soluble polymers in aqueous solution, complex formation between the polymer and mucin is evident from changes in the physical state of the mixture. A mucin-mixing observational assay was therefore established to determine which polymers are capable of complexing with mucin and to classify the appearance of the resulting mixture using a simple grading scale. The physical presentation of the polymer/mucin complex provides valuable insight on the cohesive nature of the complex and is helpful in assessing whether an occlusive barrier with extended network properties may form with mucus in the intestine by oral dosing of such polymer in-vivo.

For example, in certain cases, mixing of the test polymer with mucin results in a clear solution equivalent in appearance to the two initial solutions. No complex formation is evident. In other cases, hazy or cloudy dispersions may form in which no particulate material is visible. Such dispersions are typically stable for days without aggregation or film formation. In other cases, a cloudy suspension of visible particles may result. These suspensions may settle out over time (hours, days), but the particles are generally not adherent or cohesive, and they can generally be re-suspended by mixing. In other cases, the polymer/mucin complex will form a monolithic gel or film that coats the walls of the mixing vessel. Such a complex, illustrating strong cohesive properties, is considered by the inventors to be the most favorable result, signaling that a complex has formed that is potentially capable of forming occlusive barrier layers on mucosal surfaces.

The objective of the mucin-mixing assay is to determine whether a test polymer forms an insoluble complex when mixed with soluble mucin glycoprotein under a set of standard conditions, and to observe the general properties of the resulting mixture. The assay outputs are: 1) assignment of a clarity descriptor, 2) assignment of a physical state descriptor, 3) optional comments, photographs or videos.

Isolation of a Water-Soluble Fraction of Sigma (Cat #1778) Mucin from Porcine Stomach, Type-3 (MPS-3):

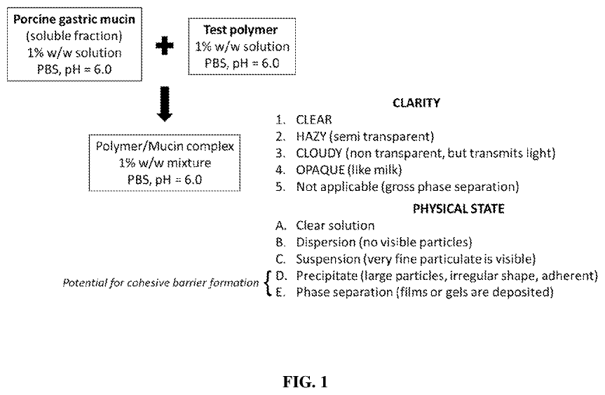

The scheme for mucin-mixing assay is shown in FIG. 1. The protocol is as follows. 1.0 g of MPS-3 is mixed with 40 ml of MilliQ deionized water in a 50 mL conical tube. The suspension was left overnight by attaching the tubes to a rotating carousel mixer. The 50 ml conical tube was then centrifuged on a Beckman Avanti centrifuge (5,300 rpm for 60 minutes). The supernatant was poured carefully into a fresh 50 ml conical tube trying to not disturb the solid pellet at the bottom. Centrifugation was then repeated on the supernatant (Beckman Avanti centrifuge, 5,300 rpm for 60 minutes). The supernatant was again collected into a tared 50 ml conical tube. The mucin solution was frozen at −80° C. for at least 2 hrs. The frozen sample was placed on a lyophilizer for at least 3-days. The lyophilized product was collected, the tube weighed, and % yield calculated, with an expected recovery of 0.60-0.65 g. The solid was stored at 2-5° C. in the refrigerator.

Polymer/Mucin Mixing Assay Protocol:

The scheme for the mucin mixing assay is shown in FIG. 1. A 1.0% w/w solution was made of each test polymer using PBS (Gibco 20012-027), pH adjusted to 6.0. A 1.0% w/w solution of water-soluble MPS-3 was made using PBS (Gibco 20012-027), pH adjusted to 6.0. The mucin solution was slightly hazy. 0.25-0.5 mL of the MPS-3 solution was placed into a 2-dram (4 mL) vial. An equivalent volume of the test polymer solution was slowly added to the vial. The vial capped and slowly rotated to mix while observing any physical changes. One of the following descriptors for the clarity of the mixtures can be selected to read the resulting mixture: clear (only slight hazy, similar to initial mucin solution); hazy (More hazy than initial mucin solution, can read text through it); cloudy (non-transparent, can't read through it, but transmits light well); opaque (like milk, no transparency, does not transmit much light) and not applicable (material shows gross phase separation). One of the following descriptors on the physical state of the mixtures can be selected to read the resulting mixture: clear; dispersion (no visible particles); suspension (very fine particulate is observable), precipitate (large irregular particles, may be adherent) and phase separation (films, gels are deposited, a clear or hazy supernatant fluid may be seen). The mixtures in capped vials were observed for approx. 1 hour to note any changes.

Results of Mucin-Mixing Assay:

Chemical modification of PAAn (Table 1) revealed that addition of hydrophobic substituents could in some cases increase the tendency to form insoluble complexes. Many modified PAAn derivatives produced cloudy particulate suspensions (physical state “B” or “C”) when mixed with mucin. Unexpectedly, the inventors found that phenyl boronic acid substitution on PAAn resulted in the formation of cohesive and adherent gels and films that were consistently found to coat the wall of the test vial (physical state “E”). This behavior was seen using amide-formation chemistry with carboxyphenylboronic acid (3-CPBA and 4-CPBA) as well as for the products of Michael addition with 3-acrylamidophenyl boronic acid (APBA). Interestingly when PAAn-4-CPBA was further modified using glycidol or modified by guanidinylation, this cohesive mucin complex formation persisted.

TABLE 1

| | Molar | | | |
|---|---|---|---|---|
| | degree of | | | |
| Modification | modi- | | Physical | |
| of PAAn | fication | Clarity | State | Comments |
| none | NA | 2 | B | Hazy dispersion, no |
| | | | | visible particulates |
| amide of hydro- | 20% | 3 | B | Cloudy dispersion, |
| caffeic acid | | | | no particulate |
| amide of | 20% | 3 | C | Cloudy, fine |
| gallic acid | | | | particulates |
| amide of | 41% | 4 | C | Opaque with small |
| benzoic acid | | | | particulates in solution |
| amide of 4-CPBA | 13% | 5 | E | Sticky gelatinous film |
| amide of 4-CPBA | 41% | 5 | E | Sticky gelatinous film |
| amide of 3-CPBA | 37% | 5 | E | Stringy complex |
| | | | | coated walls |
| Michael addition | 10% | 5 | E | Stringy gel |
| of APBA | | | | |
| Michael addition | 25% | 5 | E | Stringy gel |
| of APBA | | | | |
| Michael addition | 50% | 5 | E | Stringy adherent |
| of APBA | | | | precipitate on walls |
| | | | | and in suspension |
| epoxide addition, | 30% | 3 | B | Cloudy dispersion, no |
| 1-epoxybutane | | | | visible particulates |
| epoxide addition, | 18% | 3 | B | Cloudy dispersion, no |
| 1-epoxyhexane | | | | visible particulates |
| epoxide addition, | 5% | 2 | B | Slightly hazy, |
| 1-epoxyoctane | | | | no particulates |
| epoxide addition, | 40% | 3 | C | Cloudy, with particles |
| 1-epoxyoctane | | | | |
| epoxide addition, | 100% | 3 | B | Cloudy suspension, |
| glycidol | | | | no particulates |
| guanidinylation | 90% | 4 | B | opaque dispersion, |
| | | | | no particles |
| amide of 4-CPBA, | 13%, 50% | 5 | E | Adherent precipitate |
| mod with glycidol | | | | |
| amide of 4-CPBA, | 13%, 80% | 5 | E | Sticky clumps of |
| guanidinylated | | | | stringy solid |
| amide of 4-CPBA, | 13%, 40% | 5 | E | Sticky clumps of |
| guanidinylated | | | | stringy solid |

Copolymers of acrylamidophenyl-3-boronic acid (APBA) with several water-soluble acrylamide monomers were tested in the mucin-mixing assay (Table 2). The charge of the APBA copolymers was varied so that neutral, anionic and cationic examples could be tested. For anionic co-monomers, 2-acrylamido-2-methylpropane sulfonic acid (AMPS) and acrylic acid (AA) were used. For neutral monomers, hydroxyethylacrylamide (HEAm), N,N-dimethyl acrylamide (DMAm) and hydroxypropylmethacrylamide (HPMA) were used. For cationic copolymers methacrylamido-3-propylamine (MAPAn), acrylamido-3-propyl-dimethylamine (APDMAn), acrylamido-3-propyl trimethylammonium chloride (APTAC), 1-vinylimidazole (1-VI), and 4-vinylpyridine (4-VP) were used.

Table 2 shows that for the boronic acid containing copolymers, only polymers bearing cationic charge formed mucin complexes, and several produced complexes with apparent extended network properties, as a result of the combined presence of a boronic acid group and a cationic group.

TABLE 2

| | | | Phy- | |
|---|---|---|---|---|
| | | Clar- | sical | |
| APBA Copolymer | Charge | ity | State | Comments |
| Poly(AMPS-co-APBA) | anionic | 1 | A | No visible change |
| 92.5/7.5 | | | | |
| Poly(AMPS-co-APBA) | anionic | 1 | A | No visible change |
| 85/15 | | | | |
| Poly(AMPS-co-APBA) | anionic | 1 | A | No visible change |
| 70/30 | | | | |
| Poly(AA-co-APBA) | anionic | 1 | A | Clear solution |
| 70/30 | | | | |
| Poly(HEAm-co-APBA) | neutral | 1 | A | No visible change |
| 80/20 | | | | |
| Poly(DMAm-co-APBA) | neutral | 1 | A | No visible change |
| 90/10 | | | | |
| Poly(HPMA-co-APBA) | neutral | 1 | A | No visible change |
| 92.5/7.5 | | | | |
| Poly(HPMA-co-APBA) | neutral | 1 | A | No visible change |
| 85/15 | | | | |
| Poly(HPMA-co-APBA) | neutral | 1 | A | No visible change |
| 70/30 | | | | |
| Poly(MAPAn-co-APBA) | cationic | 2 | B | Hazy dispersion |
| 92.5/7.5 | | | | |
| Poly(MAPAn-co-APBA) | cationic | 2 | B | Hazy dispersion |
| 85/15 | | | | |
| Poly(MAPAn-co-APBA) | cationic | 5 | E | Adherent |
| 70/30 | | | | opaque gel. |
| Poly(1-VI-co-APBA) | cationic | 5 | E | Stringy |
| 80/20 | | | | precipitate |
| | | | | stuck to wall |
| Poly(APDMAn-co-APBA) | cationic | 2 | B | Hazy dispersion |
| Poly(APTAC-co-APBA) | cationic | 2 | C | Hazy suspension, |
| 70/30 | | | | fine particles |
| Poly(4-VPy-co-APBA) | cationic | 3 | C | Cloudy |
| 80/20 | | | | suspension |

An assay for the primary assessment of complex formation with mucin in aqueous solution has been utilized for the screening of a set of synthetic polymers as mucin-interacting agents. The assay outputs are assignment of a clarity descriptor, and assignment of a physical state descriptor. The assay is capable of distinguishing between compounds that are fully miscible with mucin from those that form dispersions or particle suspensions, and clearly identify compounds capable of forming complexes with extended network properties.

The complete precipitation of mucin into a cohesive mass (phase separation) was only observed for certain polycation derivatives bearing a phenylboronic acid group (4-CPBA or 3-CPBA). Amides of 4-CPBA with poly(allylamine) were particularly robust, demonstrating phase separated mucin/polymer complexes even when the level of CPBA substitution was reduced to 5 mol %. The formation of the cohesive mucin complex with PAAn-4-CPBA was pH dependent, being inhibited at pH=2, and appearing to build robustness with an increase in pH. A complementary result was seen for copolymers of 3-acrylamidophenylboronic acid (APBA). It was found that only certain cationic copolymers of APBA were capable of forming phase-separated, cohesive mucin complexes.

### III. Centrifuge Assay

The scheme for the centrifuge assay is shown in FIG. 2. When polymer-mucin complexes are formed in the cup of a centrifuge filter device and subjected to centrifugation under a standard set of conditions, the fluid mixture is passed through the filter membrane under centrifugal force. However, this filtration process can be inhibited in cases where the polymer-mucin complex has developed physical properties that are not solution-like. In these cases, a significant portion of the mixture may be retained in the filter cup. The fraction of retained material can be measured gravimetrically and correlated with the physical properties of the polymer-mucin complex.

A set of experimental conditions capable of effecting complete filtration for solutions of mucin or polymer alone can be defined. However, when mucin and polymer solutions are mixed together in the filter cup forming a complex, centrifuge filtration may be inhibited to various extents depending on the physical properties of the polymer-mucin complex. The inventors have found that in some cases, cloudy dispersions of polymer-mucin complex may be completely passed through the filter. In contrast, polymer-mucin complexes with gel-like or other complex morphologies may be strongly retained in the filter cup. Material is retained in the filter cup only in cases where the polymer/mucin complex has developed adherent, gelatinous, or other non-solution physical properties.

The inventors believe that this simple assay is capable of differentiating between polymer-mucin complexes that remain flowable and well dispersed in solution, from those exhibiting viscoelastic physical properties and an extended network structure. It is proposed that this resistance to filtration may correlate positively with the polymer's ability to condense mucus into an occlusive barrier material.

The objective of the centrifuge assay is to mix a test polymer with a soluble mucin glycoprotein in the cup of a centrifuge filter device and to quantify the mass of material retained in the filter cup after centrifugation under a set of standard conditions. The assay output is fraction retained in the filter cup (Fraction Retained, or % R).

Centrifuge-Filter Assay Protocol:

A 1.0% w/w solution of each test polymer using PBS (Gibco 20012-027) was made and pH to 6.0. A 1.0% w/w solution of water-soluble MPS-3 using PBS (Gibco 20012-027) was made and pH to 6.0. The mucin solution was slightly hazy. The pH of all solutions were adjusted to 6 with 1 M NaOH and 1 M HCl. COSTAR, Spin-X Centrifuge Tube Filters, 0.45 μm Cellulose Acetate, 2 ml tube, non-sterile (Corning Inc.) was obtained. Each centrifuge filter tube was labelled and the mass of each tube, without the filter cup was recorded. This was the pre-filtration tare weight of the tube. 75 μL mucin solution was placed into each filter cup, making sure that the filter is completely coated. 75 μL of the test polymer solution was placed into the filter cup. The desired number of replicates for each polymer (typically n=4), and for a blank with 75 μL mucin solution and 75 μL PBS adjusted to pH=6.0 were replicated. All centrifuge cups contain 150 μL of solution, and assumed to weigh 150 mg. All tubes were placed in an incubating mini shaker at 37° C. and 300 rpm for 30 minutes. The tubes were placed in a Beckman Microfuge-R centrifuge (or similar instrument) and centrifuged at 6000 times gravity for 12 minutes at 21° C. The tubes were collected and carefully weighed after removing the filter-cup. This was the post-filtration tube weight. The weight of fluid filtered into the tube was calculated by subtracting the pre-filtration tube weight from the post-filtration tube weight. The fraction of material retained (Fraction Retained, or fR) was calculated in the cup by subtracting the weight of the filtrate from the total weight of the polymer-mucin mixture (0.150 g) and dividing by 0.150. The average value, standard deviation, and number of replicates for the “Fraction Retained” endpoint can be calculated.

Chemical modifications of polycations such as poly(diallylamine) (PDAAn), poly(ethyleneimine) (PEI), poly(methacrylamidopropylamine) (PMAPAn) and poly(allylamine) (PAAn) produced amide derivatives which in some cases displayed greater propensity for forming insoluble complexes with mucin with extended network structure properties as seen by their resistance to filtration (Table 3). This result is consistent with observations of gross phase separation made upon mixing these polymers with mucin in solution. In general, polymers that gave a larger amount of retained material in the centrifuge assay formed phase separated precipitates that were visually distinguishable from the cloudy dispersions or suspensions formed by the unmodified parent polycation. Of particular note were the amides of 4-carboxyphenyl boronic acid, which consistently performed well (fR from 0.42 to 0.70).

TABLE 3

| | | St. | |
|---|---|---|---|
| Polymer | fR | Dev. | Replicates |
| PDAAn | 0.05 | 0.004 | n = 4 |
| PDAAn Gallic amide | 0.05 | 0.017 | n = 4 |
| PDAAn Hydrocaffeic amide | 0.38 | 0.081 | n = 4 |
| PDAAn 4CPBA amide | 0.42 | 0.026 | n = 3 |
| PEI | 0.01 | 0.005 | n = 4 |
| PEI Gallic amide | 0.25 | 0.128 | n = 4 |
| PEI Hydrocaffeic amide | 0.22 | 0.022 | n = 4 |
| PEI 4CPB amide | 0.42 | 0.040 | n = 3 |
| PMAPA | 0.05 | 0.010 | n = 4 |
| PMAPA Gallic amide | 0.02 | 0.017 | n = 4 |
| PMAPA Hydrocaffeic amide | 0.40 | 0.037 | n = 4 |
| PMAPA 4CPBA amide | 0.69 | 0.033 | n = 4 |
| PAAn | 0.03 | 0.013 | n = 4 |
| PAAn-Gallic amide | 0.43 | 0.025 | n = 3 |
| PAAn Hydrocaffeic amide | 0.46 | 0.083 | n = 4 |
| PAAn-4CPBA (30% Substitution) | 0.70 | 0.021 | n = 4 |

Evaluation of Copolymers in the Centrifuge Assay:

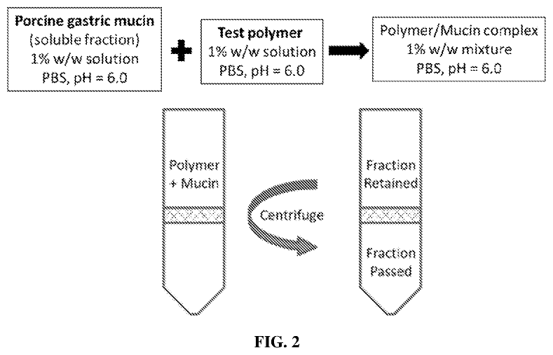

A simple, commercially available, polymerizable phenylboronic acid is 3-acrylamidophenyl boronic acid (APBA). APBA itself is not water soluble, and its homopolymer is also not water soluble. APBA copolymers with acrylamide monomers were evaluated for their potential as mucus-interacting agents (FIG. 3). APBA copolymers were prepared with neutral (2-hydroxypropyl-methacrylamide, HPMA), anionic (2-Acrylamido-2-methyl-1-propanesulfonic acid, AMPS), and cationic ((3-methacrylamido)propylamine, MAPAn) co-monomers. Within this set of copolymers, the molar ratio of APBA was increased from 7.5% to 15% to 30%.

All copolymers were found to be soluble with the exception of the neutral copolymer with the highest APBA content. When mixed with mucin in PBS (pH=6) at 1 wt %, the neutral and anionic copolymers gave clear solutions with no sign of complex formation. In contrast, the cationic MAPAn copolymers gave cloudy dispersions for the 7.5% and 15% APBA compositions. The 30% APBA copolymer produced gross phase separation and a white adherent precipitate. FIG. 3 and Table 4 show the results of the centrifuge assay evaluation for this set of copolymers.

TABLE 4

| Polymer | fR | St. Dev. | Replicates | |
|---|---|---|---|---|
| Poly(AMPS-co-APBA) 70/30 | 0.04 | 0.021 | n = 4 | Anionic |
| Poly(AMPS-co-APBA) 85/15 | 0.03 | 0.020 | n = 4 | Anionic |
| Poly(AMPS-co-APBA) 92.5/7.5 | 0.04 | 0.018 | n = 4 | Anionic |
| Poly(MAPA-co-APBA) 70/30 | 0.63 | 0.038 | n = 4 | Cationic |
| Poly(MAPA-co-APBA) 85/15 | 0.07 | 0.018 | n = 4 | Cationic |
| Poly(MAPA-co-APBA) 92.5/7.5 | 0.06 | 0.057 | n = 4 | Cationic |
| Poly(HPMA-co-APBA) 70/30 | insoluble | | | Neutral |
| Poly(HPMA-co-APBA) 85/15 | 0.00 | 0.031 | n = 4 | Neutral |
| Poly(HPMA-co-APBA) 92.5/7.5 | 0.02 | 0.025 | n = 4 | Neutral |

Four additional cationic copolymers were prepared with APBA and tested in the centrifuge assay. These included (3-acrylamidopropyl)dimethylamine (APDMAn), (3-acrylamidopropyl)trimethylammonium chloride (APTAC), 4-vinylpyridine (4-VPy) and 1-vinylimidazole (1-VI). The results of the centrifuge assay for these four materials along with the cationic ((3-methacrylamido)propylamine, MAPAn) copolymer are shown in FIG. 4.

For all cationic copolymers of APBA, mucin complexation resulted in significant retained material in the centrifuge assay. It was also consistently observed that the cationic homopolymer without APBA did not score well in the assay. As with the modified polyamines of Table 3, a positive result in the centrifuge assay (generally fR>0.4) was consistent with the observation of gross phase separation when the polymer was mixed with mucin in dilute solution.

### IV. Multi-Particle Tracking (MPT) Assay

Real-time multi-particle tracking (rtMPT) is a powerful method for measuring diffusion through gastrointestinal mucus. rtMPT is a well-established technique that has been utilized in a number of drug and gene carrier transport studies. rtMPT uses video microscopy to track the microscopic motion of hundreds of individual polystyrene nano-particles simultaneously with high-spatiotemporal resolution. Analysis of both individual and ensemble particle data reveal important insights into particle-environment interactions and bulk as well as micro-transport properties. Quantitative and qualitative information such as diffusivity, viscoelasticity, pore size, velocity, directionality, and transport mode can be determined from the particle trajectories.

The inventors have studied the effect of the mucus-binding polymers of the invention on the barrier properties of fresh porcine intestinal mucus using rtMPT. Fluorescent polystyrene nanospheres 200 nm in diameter with PEGylated surfaces were used as the model diffusion probes. Surface PEGylation provides readily diffusible particles with no surface charge and no significant chemical interactions with mucus or polymer samples. Diffusion of these “stealth” particles was studied in native small intestinal porcine mucus. Particle trajectories were used to calculate time-averaged mean squared displacements (MSDs), which were subsequently used to determine time-dependent diffusion coefficients and to characterize individual particle transport modes.

Multi-Particle Tracking Assay Protocol: Intestinal Mucus Collection:

Native porcine small intestinal mucus was harvested and rinsed with cold water to remove bulk material at local abattoir (Research 87 Inc., Boylston Mass.). The mucus layer was gently scraped using a metal spatula, and then stored in microcentrifuge tubes at −80° C. Frozen mucus was thawed for 30 minutes at room temperature prior to use.

Nanoparticle Preparation and Characterization:

1-Ethyl-3-(3-dimethylaminopropyl)-carbodiimide (EDC, Sigma) cross-linking chemistry was used to covalently conjugate 2000 molecular weight (MW) amine-terminated PEG (PEG-NH4, Laysan Bio, Inc.) to carboxyl-modified particles (FluoSpheres®, Life Technologies). Briefly, the carboxyl-modified particles were diluted in 50 mM 2-(N-morpholino) ethanesulfonic acid (MES buffer, pH 6.5, Sigma), then 20 mg PEG-NH4 and 10 mg EDC were added to the particle solution and mixed for 2 hrs at room temperature. To quench the reaction, 100 mM glycine (Sigma) was added and mixed for 30 mins. The particle solution was dialyzed overnight using Slide-A-Lyzer® Mini Dialysis Devices, 10 kDa cut off (ThermoFisher Scientific).

Nanoparticle and Mucus Sample Preparation:

Transport properties of 200 nm fluorescent particles with PEG surface functionalization were probed using multiple particle tracking technique. To prepare particle suspensions, the particles were diluted to a final concentration of 0.0025% wt/vol in the presence of 1% polymer solution, or water as control. Fresh porcine intestinal mucus (150 μL) was added to a Lab-Tek® Chamber Slide™ (Nunc™). Particle suspensions (7.5 μL, PEG-modified particles) in 1% polymer solution, or water (control) were vortexed and added drop-wise with minimal perturbation to the mucus surface. The sample was incubated in a dark, humidified chamber for 2 hrs at room temperature prior to imaging.

Multiple Particle Tracking:

Particle videos were obtained using an X-Cite 120 fluorescence illumination system and Olympus DP70 digital color camera attached to an inverted Olympus IX51 microscope. Briefly, 20 sec trajectory videos were recorded with frame rate of 30 frames per second. Particle trajectories were analyzed using custom MATLAB software previously developed in the research group of Prof. Rebecca Carrier at Northeastern University (Boston, Mass.) to calculate mean-squared displacement and effective diffusivity (Deff). The experiments were carried out in accord with well-established protocols as described in the published literature [Lock, J. Y., Carlson, T. L., Wang, C.-M., Chen, A. & Carrier, R. L. Acute Exposure to Commonly Ingested Emulsifiers Alters Intestinal Mucus Structure and Transport Properties. Sci. Rep. 8, 10008 (2018).].

Polyallylamine (PAAn) and poly(methacrylamidopropylamine) (PMAPAn) modified with 4-carboxyphenylboronic acid (4-CPBA) were tested in the rtMPT assay (Table 5), along with relevant control and comparison compounds.

Results of Real-Time Multi-Particle Tracking Assay:

Poly(vinylpyrrolidone), a neutral mucoadhesive polymer was used as a control. The relative Deff value for unmodified poly(allylamine) was 23% of the same day control (a 4.4× reduction). For poly(allylamine) modified with glycidol, a similar result was found (27% of the same day control, a 3.8-fold reduction in Deff). For poly(allylamine) modified with carboxyphenyl-4-boronic acid, an even lower diffusion coefficient was obtained (9% of the same-day control, or an 11-fold reduction in Deff).

For poly(methacrylamidopropylamine) (PMAPAn), the unmodified polycation gave a Deff value that was 17% of the same day control (a 6-fold reduction). In comparison, the carboxyphenyl-4-boronic acid modified polymer gave a Deff value that was only 1% of the same day control (a 100-fold reduction in Deff).

TABLE 5

| | Deff (μm²/s) | | |
|---|---|---|---|
| | with | without | relative |
| Polymer | polymer | polymer | Deff |
| Poly(vinylpyrrolidone) | 0.0148 | 0.0137 | 108% |
| Poly(allylamine) (PAAn) | 0.0083 | 0.0366 | 23% |
| PAAn-Glycidol (80% mod.) | 0.0097 | 0.0366 | 27% |
| PAAn-CPBA (30% mod.) | 0.0033 | 0.0366 | 9% |
| Poly(methacrylamidopropyl- | 0.00387 | 0.0231 | 17% |
| amine) (PMAPAn) | | | |
| PMAPAn-CPBA (25% mod.) | 0.000247 | 0.0231 | 1% |

### V. Chronic Rat Studies

A chronic rat study was conducted in order to investigate metabolic improvement resulting from 60-day daily dosing of poly(allylamine hydrochloride) (PAAn-HCl) modified with 4-carboxyphenylboronic acid (CPBA).

Chronic GK rodent study was conducted using the poly(allylamine) derivative of EXAMPLE 32 by daily administration over an 8-week period in Goto-Kakazaki (GK) rats. Animals were allowed to acclimate for at least one week and housed at 19° C.-22° C. and 40%-60% humidity with a 12-hour light-dark cycle. At the beginning of the study, rats were randomized into two groups: a control group receiving 0.9% saline (n=7), and a polymer treatment group receiving polymer (80 mg/ml) dissolved in saline (n=7). Throughout the 8-week study period, control and treatment solutions were administered by oral gavage once per day in a 1.5 mL bolus via oral gavage. Rats were fasted daily from 2:00-5:00 PM with free access to water. Gavage dosing was performed once per day at 6:00 PM.

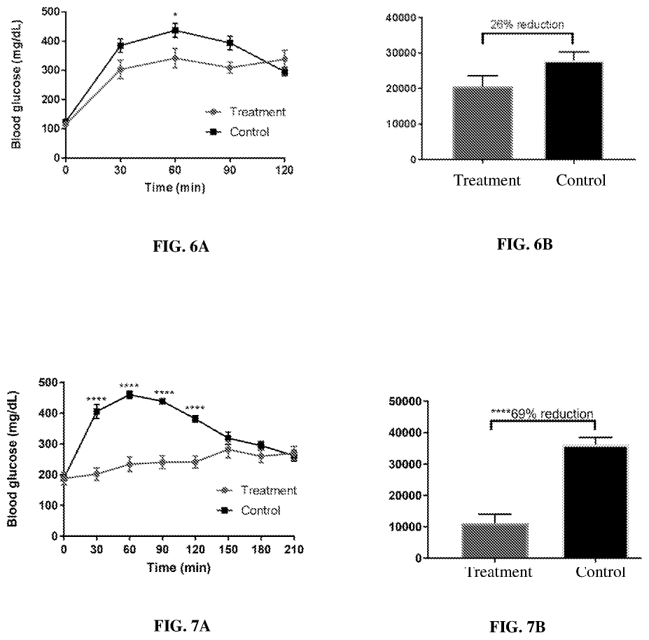

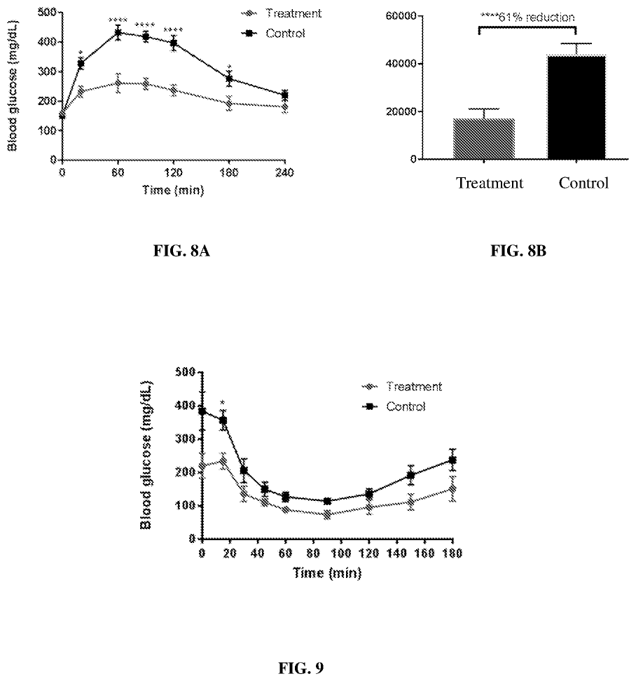

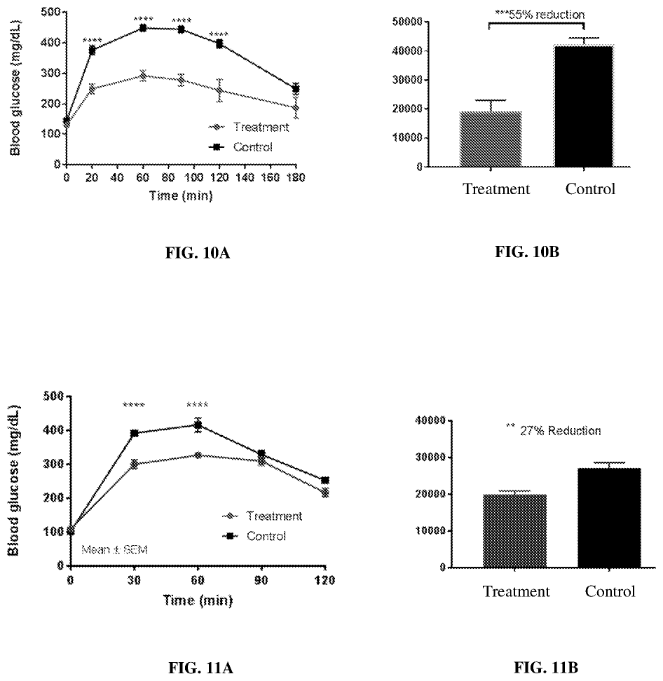

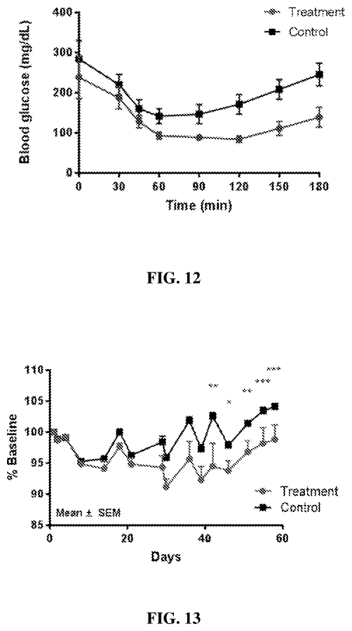

Metabolic testing was conducted several times throughout the 8-week study. Three categories of tests were used: (1) Oral glucose tolerance test (oGTT) on Study Day 8 (FIGS. 6A-6B), Day 30 (FIGS. 7A-7B), Day 39 (FIGS. 8A-8B), and Day 52 (FIGS. 11A-11B), (2) Mixed meal tolerance test (mmTT) on Study Day 46 (FIGS. 10A-10B), and (3) Insulin tolerance test (ITT) on Study Day 42 (FIG. 9) and Day 55 (FIG. 12).

The primary endpoint of the study was the reduction in the incremental area under the blood glucose curves at each testing day, which was compared to one another at each time point and for an overall significance value.

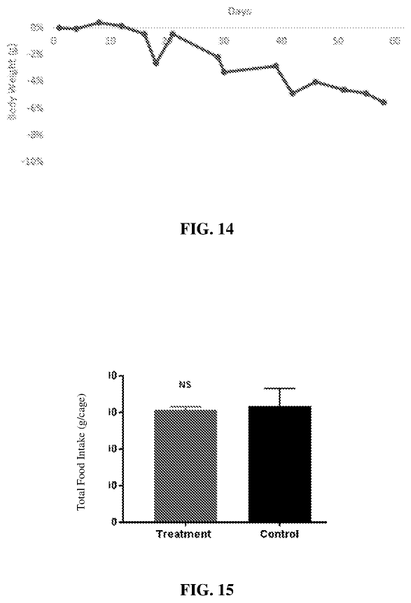

Secondary endpoints of the study included weight gain profiles and Homeostatic Model Assessment of Insulin Resistance (HOMA-IR) (FIG. 13 and FIG. 14).

On the night prior to a planned oGTT or mmTT, all rats were fasted from food in hanging cages overnight for 12-16 hours and allowed water ad libitum. On the morning of the study following the fast, rat tails were slit to enable blood glucose measurement, and baseline blood glucose was measured for all animals. Oral gavage of test article was then administered. One hour later, an oral gavage of a 40% glucose solution (2.0 g/kg body weight) was administered. Blood glucose measurements were then taken from each animal at 0, 30, 60, 90, 120, and optionally at 180, 210, and 240 minutes after the glucose administration. Blood glucose values from each group at each time point were calculated and plotted to profile oGTT curves and a comparison between control and treatment groups was made.

In the case of ITT, preparation for the study differed in that no fasting was performed and insulin was injected intraperitonially at 0.75 U/kg rat (instead of glucose dosed orally). The remainder of the tolerance test study was performed as described with blood glucose measurements being taken from the tail over the 2-hour period.

Chronic once daily administration of PAAn-HCl modified with CPBA demonstrated a robust and statistically significant effect of glycemic homeostasis in this well-validated GK rat model. Data is presented for each tolerance test as Mean±SEM with a one-way ANOVA followed by multiple comparisons. Significance is denoted as:

*p<0.05, **p<0.005, ***p<0.001, and ****p<0.0001.

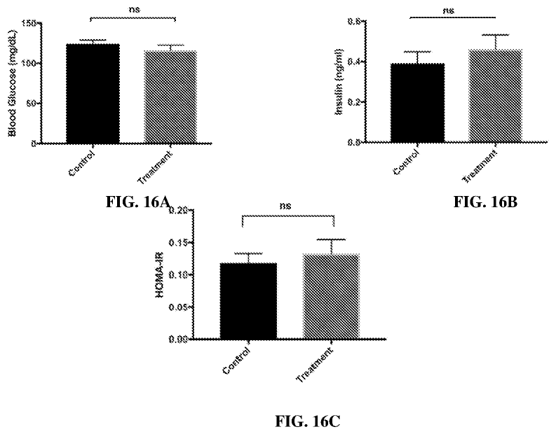

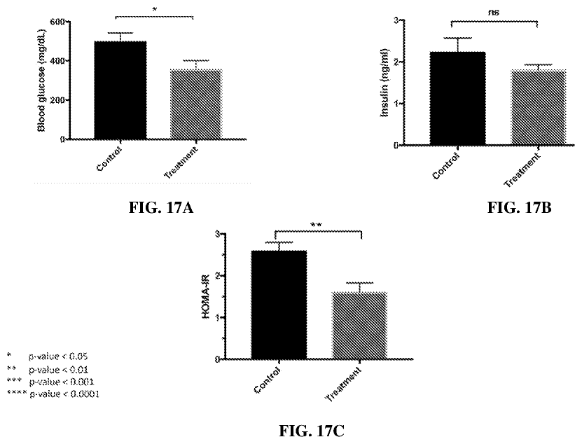

The chronic GK rodent study demonstrates the efficacy and tolerability of the poly(allylamine) derivative of EXAMPLE 32 over an 8-week period (FIGS. 16A-16C and FIGS. 17A-17C).

There was a significant enhancement of the poly(allylamine) derivative's effect on glucose homeostasis over time. This is consistent with the observation that chronic duodenal exclusion results in metabolic resetting towards an improved systemic metabolic milieu. Also, the effect on fasting glycemia suggests that chronic polymer treatment (based on ITT results) stimulates a persistent change in systemic glucose homeostasis, likely via suppression of hepatic glucose production. However, in spite of disparate fasting glycemia, the ITT curves suggest that normal counter-regulatory responses are maintained. Altogether, these data suggest that chronic treatment with the poly(allylamine) derivative stimulates a profound enhancement in glucose tolerance that appears to be independent of systemic enhancements in insulin action, but may include enhanced glucose-stimulated insulin secretion and suppression of hepatic glucose output.

We hypothesize that a continued improvement in insulin resistance and additional loss of weight in the treatment group would be seen if the study were to be continued further. The weight loss of 6% observed in our chronic study is a significant observation because the GK-rat is a lean diabetic phenotype. This magnitude of weight loss is equivalent to that observed with SGLT2 inhibitors in the GK-rat model under similar experimental conditions (Takasu T, et al, Biological and Pharmaceutical Bulletin, 40(5), pp. 675-680, 2017). Interestingly, this same SGLT2 inhibitor resulted in ˜15% total body weight loss in an obese rat model (Yokono M, et al, European Journal of Pharmacology, 727, pp. 66-74, 2014). It is also important to note that we did not see a difference in food intake between the control and treatment group with the poly(allylamine) derivative (FIG. 15). This is in strong contrast to SGLT2 inhibitors which increase food intake due to glycosuria.

While the invention has been particularly shown and described with reference to specific preferred embodiments, it should be understood by those skilled in the art that various changes in form and detail may be made therein without departing from the spirit and scope of the invention as defined by the appended claims.

1. A polymer comprising a repeat unit of repeat unit of any one of Formulas (I)-(III), 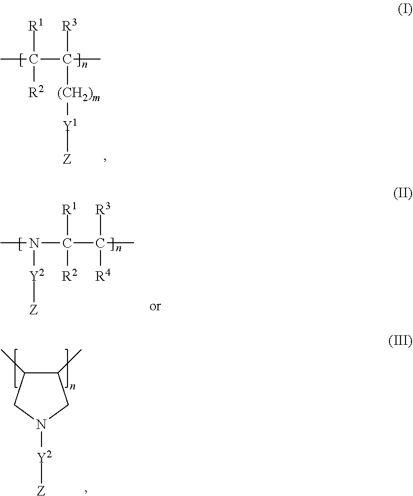
wherein,
R¹, R², R³ and R⁴ are independently hydrogen or substituted or unsubstituted alkyl;
Y¹ in each occurrence is independently a direct bond or -L¹-A¹-L²-A²-;
L¹ in each occurrence is —NR⁹—, —NC(O)— or —C(O)N—;
L² in each occurrence is absent, —NR⁹—, —O— or —S—;
Y² in each occurrence is independently a direct bond or -L³-A¹-L²-A²-;
L³ in each occurrence is —C(O)— or absent;
L² in each occurrence is absent, —NR⁹—, —O— or —S—;
A¹ and A² in each occurrence are independently absent or optionally substituted C₁-C₅ alkylene;
R⁹ in each occurrence is independently hydrogen or substituted or unsubstituted alkyl;
Z in each occurrence is 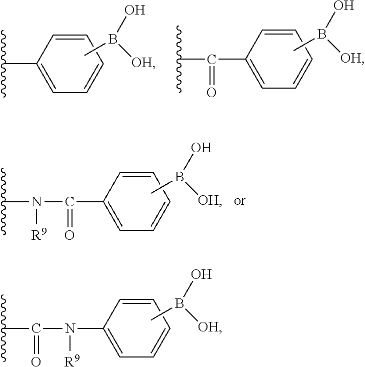
and preferably the
—B(OH)₂ is at the 3- or 4-position of the ring;
n is an integer from 1 to 100,000 and
m is an integer from 0 to 4; with the proviso that the polymer is not 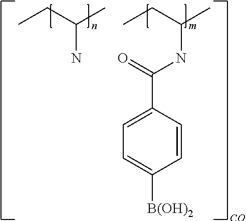
and when the polymer is 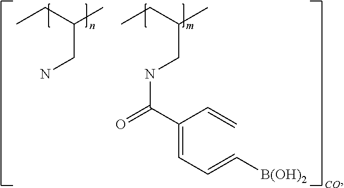
the polymer does not have a molecular weight that is greater than 100,000.

2. The polymer of claim 1, comprising a repeat unit of Formula (I).

3. The polymer of claim 2, wherein the repeat unit of Formula (I) is selected from the group consisting of 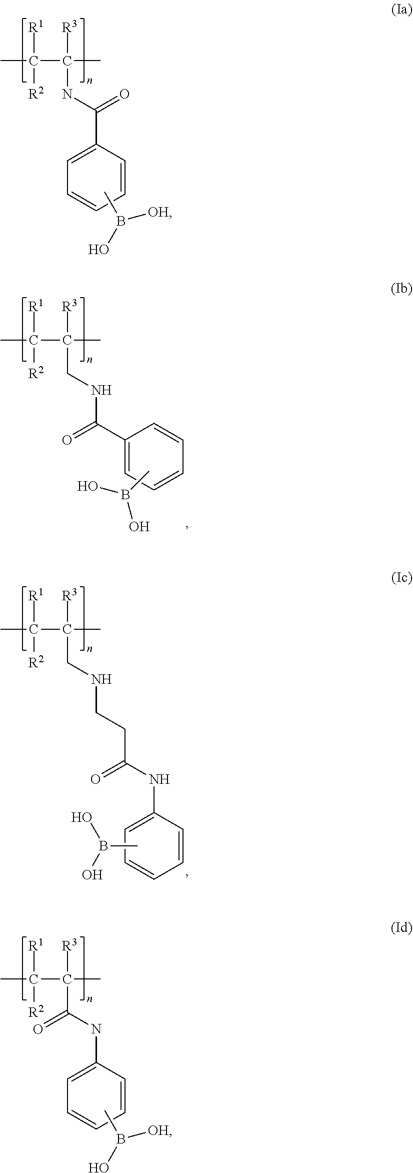 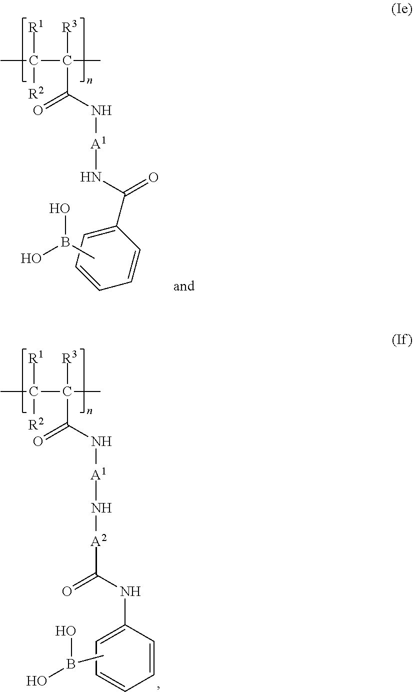
and preferably the —B(OH)₂ is at the 3- or 4-position of the ring.

4. A polymer comprising a repeat unit of Formula (Ia) to (If) according to claim 3, wherein,
R¹, R² and R³ are hydrogen;
A¹ and A² are independently absent or optionally substituted C₁-C₅ alkylene.

5. A polymer comprising a repeat unit of Formula (Ia) to (If) according to claim 3, wherein,
R¹ and R² are hydrogen;
R³ is methyl;
A¹ and A² are independently absent or optionally substituted C₁-C₅ alkylene.

6. The polymer of claim 1, comprising a repeat unit of Formula (II).

7. The polymer of claim 6, wherein in the repeat unit of Formula (II),
Y² is direct bond;
Z is 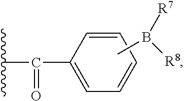
and preferably the —B(OH)₂ is at the 3- or 4-position of the ring.

8. The polymer of claim 1, comprising a repeat unit of Formula (III).

9. The polymer of claim 8, wherein in the repeat unit of Formula (III),
Y² is direct bond;
Z is 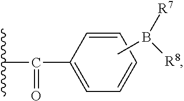
and preferably the —B(OH)₂ is at the 3- or 4-position of the ring.

10. A polymer comprising
a) At least one repeat unit selected from the group consisting of 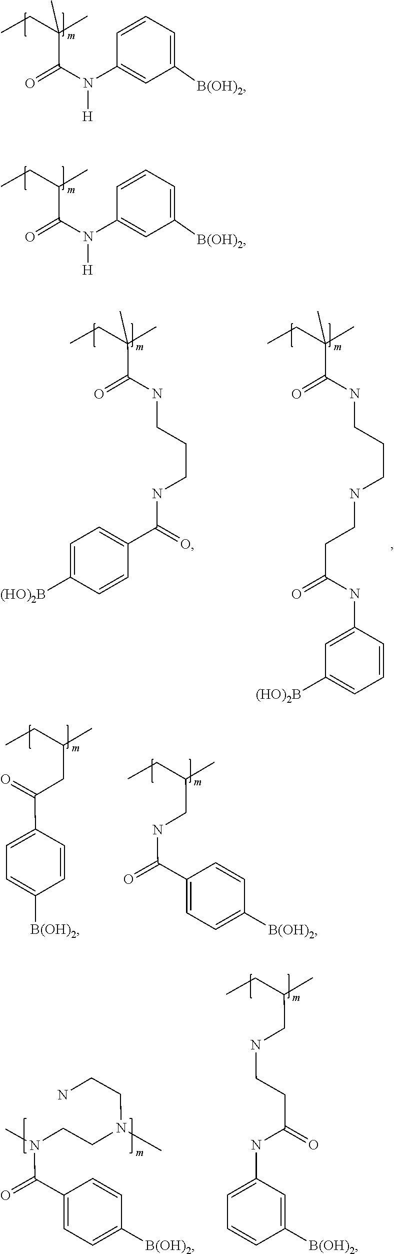 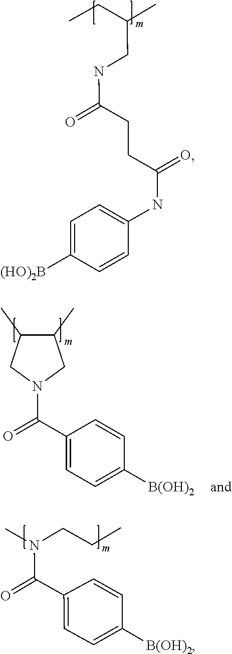
where “m” represents an integer from 1 to 100,000, and
b) At least one repeat unit selected from the group consisting of 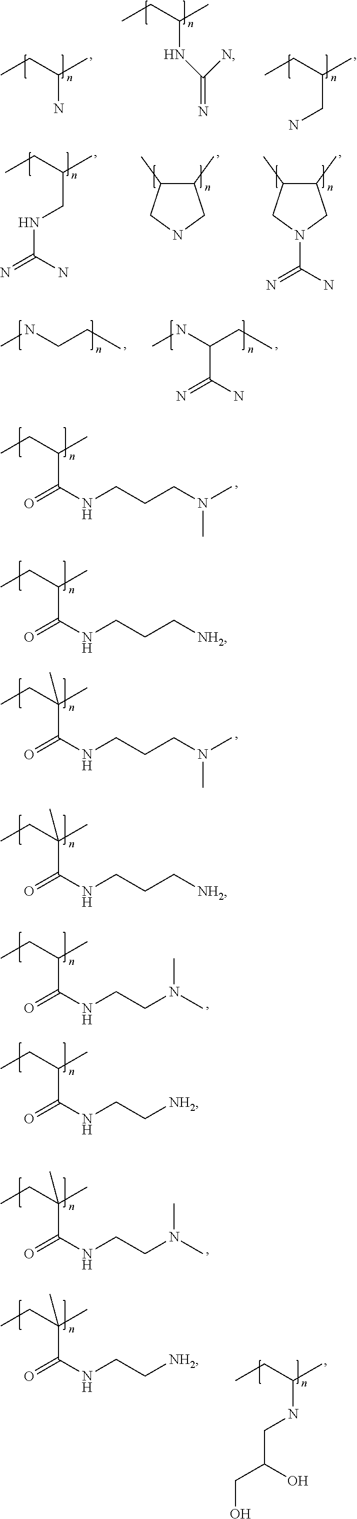 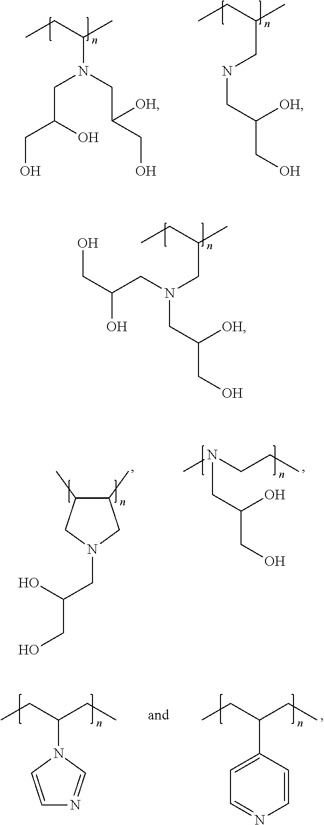
where “n” represents an integer from 1 to 100,000.

11. A polymer selected from the group consisting of 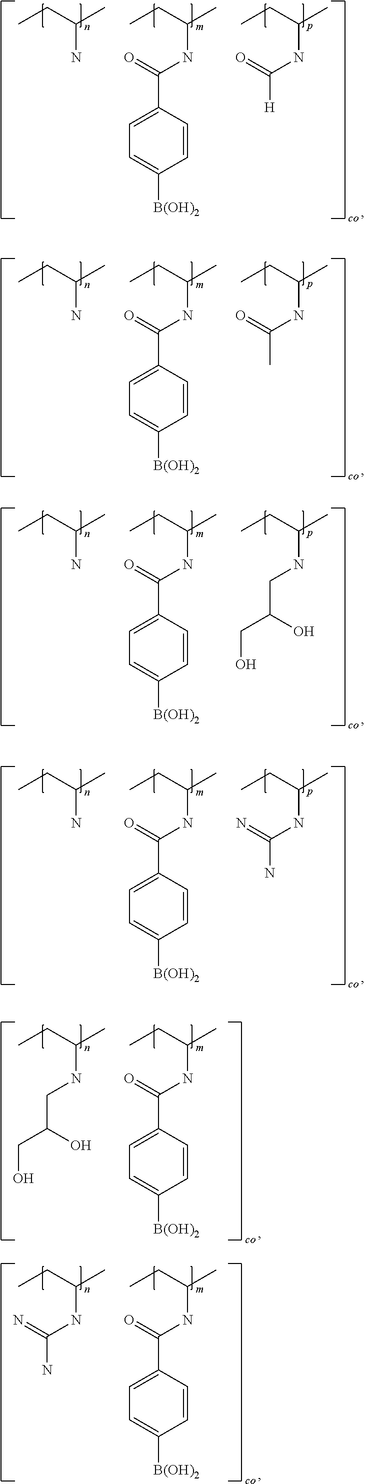 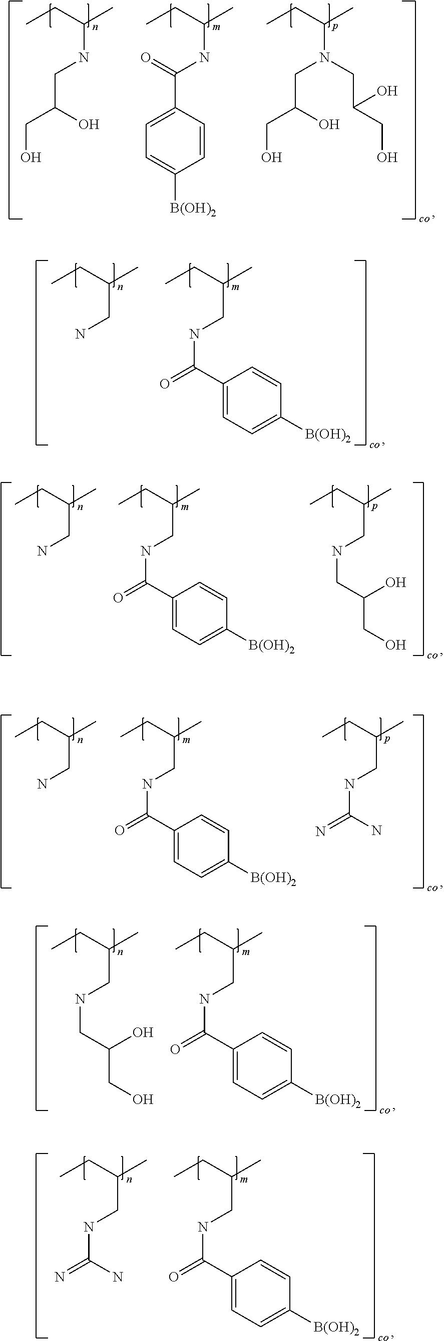 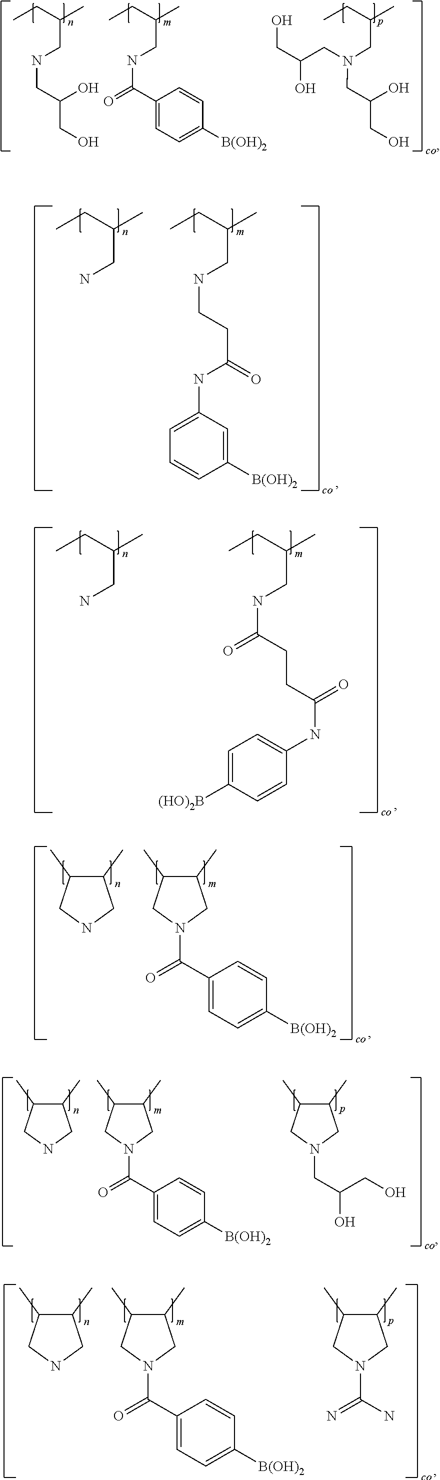 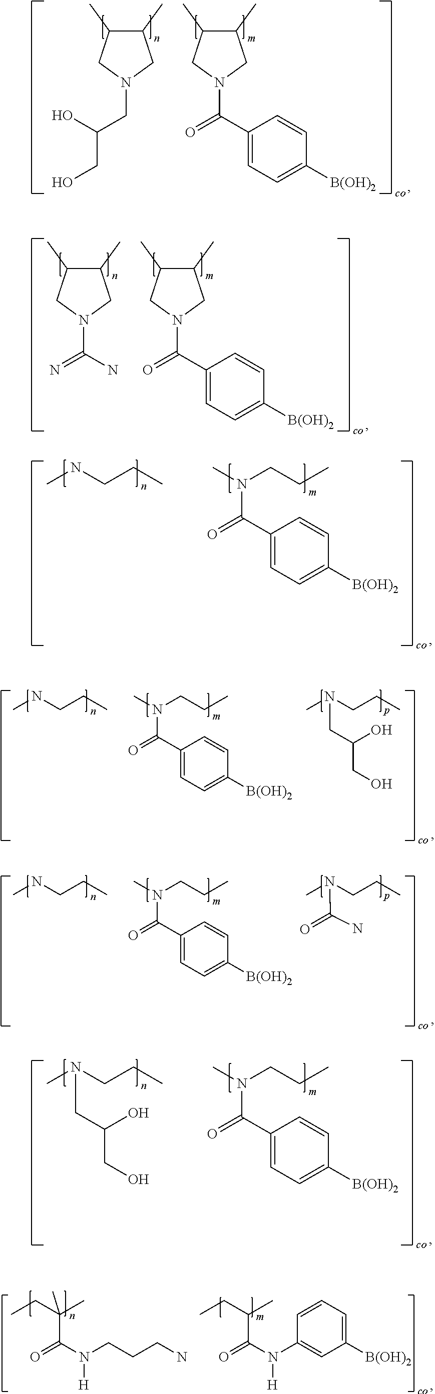 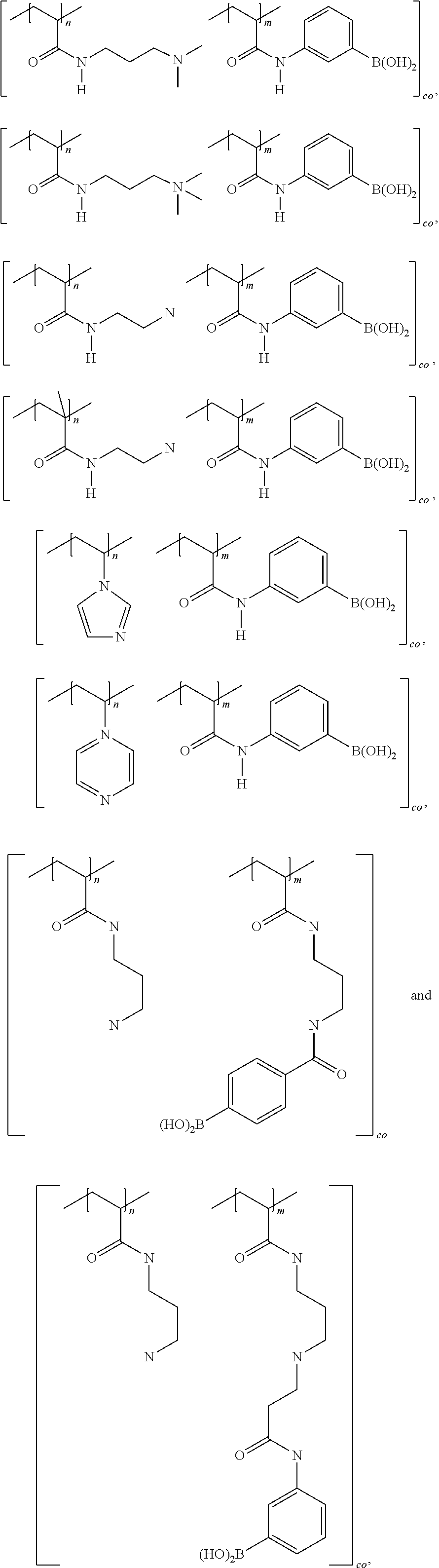
with the proviso that when the polymer is 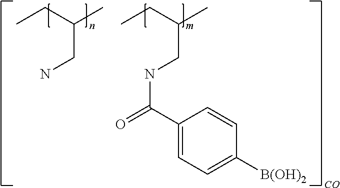
it does not have a molecular weight greater than 100,000.

12. A pharmaceutical composition comprising a polymer of claim 1 or 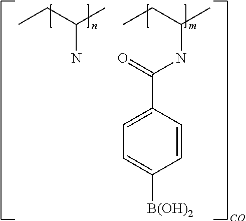
and a pharmaceutically acceptable excipient.

13. The pharmaceutical composition of claim 12, wherein the composition is a liquid, tablet, caplet or capsule.

14. The pharmaceutical composition of claim 12, wherein the composition is an enteric coated tablet, caplet or capsule.

15. The pharmaceutical composition of claim 14, wherein the enteric coated tablet, caplet or capsule is for targeted deliver to the duodenum.

16. A method for treating a metabolic disorder in a subject, comprising administering to a subject in need thereof a therapeutically effective amount of a polymer according to claim 1.

17. The method of claim 16, further comprising administering another therapeutic agent to the subject in need thereof.

18. The method of claim 16, wherein the metabolic disorder is glucose intolerance, T1DM, T2DM, prediabetes, hyperlipidemia, obesity, overweight, dyslipidemia, hypertension, hyperglycemia, impaired glucose tolerance, insulin resistance, metabolic syndrome, non-alcoholic steatohepatitis (NASH), non-alcoholic fatty liver disease (NAFLD) and polycystic ovary syndrome (PCOS).

19. The method of claim 16, wherein the metabolic disorder is type-2 diabetes mellitus.

20. A method for treating gastrointestinal disorders in a subject, comprising administering to a subject in need thereof a therapeutically effective amount of a polymer according to claim 1.
